(19) United States
(12) Patent Application Publication
(10) Pub. No.: US 2026/0202246 A1
(43) Pub. Date: Jul. 16, 2026

(54) ADVANCED SPECTROSCOPIC DEVICES

(71) Applicants: THE TRUSTEES OF THE UNIVERSITY OF PENNSYLVANIA, Philadelphia, PA (US); DREXEL UNIVERSITY, Philadelphia, PA (US)

(72) Inventors: Amanda WATSON, Charlottesville, VA (US); Claire KENDELL, Philadelphia, PA (US); Insup LEE, Newtown Square, PA (US); James WEIMER, Franklin, TN (US); Anush Niranjan LINGAMOORTHY, Philadelphia, PA (US)

(21) Appl. No.: 19/134,411

(22) PCT Filed: Nov. 30, 2023
(86) PCT No.: PCT/US2023/081940

§ 371 Date: May 30, 2025

Related U.S. Application Data
(60) Provisional application No. 63/385,507, filed on Nov. 30, 2022.

(51) Int. Cl. G01J 3/02; G01J 3/10; G06F 1/16; G06F 3/01

(52) U.S. Cl. CPC G01J 3/0264; G01J 3/0256; G01J 3/10; G06F 1/163; G06F 1/1684; G06F 3/011; G01J 2003/104

(57) ABSTRACT

Methods and devices for advanced spectroscopy are described herein. In one aspect, a method for determining levels of a biomarker of a user by a mobile device can include: generating a first light having a first wavelength; collecting emission signals resulting from the first light interacting with the user; measuring emission counts of the emission signals over a period of time; causing to be determined at least one of (i) a level of the biomarker of the user and (ii) a change in the levels of the biomarker of the user over the period of time based on the measured emission counts; and generating a notification indicative of the at least one of (i) a level of the biomarker of the user and (ii) a change in the levels of the biomarker of the user.

### CROSS-REFERENCE TO RELATED APPLICATIONS

The present application claims priority to and the benefit of U.S. patent application No. 63/385,507, “Advanced Spectroscopic Devices” (filed Nov. 30, 2022), the entirety of which application is incorporated herein by reference for any and all purposes.

### GOVERNMENT RIGHTS

This invention was made with government support under EB029363 and EB029767 awarded by the National Institutes of Health and 2125561 and 1915398 awarded by the National Science Foundation. The government has certain rights in the invention.

### TECHNICAL FIELD

The present disclosure relates to the field of spectroscopy and to wearable devices.

### BACKGROUND

Spectroscopy, the study of the interaction between electromagnetic radiation and matter, is a vital technique in many disciplines. This technique is limited to lab settings, and, as such, sensing is isolated and infrequent. Thus, it can only provide a brief snapshot of the monitored parameter. Wearable technology brings sensing and tracking technologies out into everyday life, creating longitudinal datasets that provide more insight into the monitored parameter, but existing wearable technologies have certain limitations. Accordingly, there is a long-felt need in the field for improved spectroscopy devices, in particular improved wearable spectroscopic devices.

### SUMMARY

Methods and devices for advanced spectroscopy are described herein. In one aspect, a method for determining levels of a biomarker of a user by a mobile device can include: generating a first light having a first wavelength; collecting emission signals resulting from the first light interacting with the user; measuring emission counts of the emission signals over a period of time; causing to be determined at least one of (i) a level of the biomarker of the user and (ii) a change in the levels of the biomarker of the user over the period of time based on the measured emission counts; and generating a notification indicative of the at least one of (i) a level of the biomarker of the user and (ii) a change in the levels of the biomarker of the user.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIG. 1 shows an illustration of optical spectroscopy.

FIGS. 2A-2C show user study results for monitoring glucose levels of a user according to the present disclosure. FIG. 2A shows results for a 475 nm LED; FIG. 2B shows results for a 515 nm LED; and FIG. 2C shows results for a 680 nm LED.

FIG. 3 shows an image of a prototype device according to the present disclosure.

FIGS. 4A and 4B show wearable optical spectrometers according to the present disclosure. Left: the wristband form factor is familiar and can be worn when moving around in the real-world. Right: the fingertip form factor is more suited to clinic settings and discrete measurements.

FIGS. 5A and 5B shows a theory of spectroscopy. FIG. 5A shows the theory of absorption spectroscopy. FIG. 5B shows the theory of emission spectroscopy.

FIG. 6 shows Lumos device form factors. Left: a finger clamp form factor. Right: a watch form factor.

FIG. 7 shows a block diagram of Lumos Device. The surface mount LEDs generate light and the photodetectors read the light through a medium. These readings are sent to the controller board, where the ADC samples the data and communicates it to the collection system.

FIG. 8 shows spectral responses for the LEDs and PDs of the devices described herein. Left: LED wavelengths. Right: PD wavelengths.

FIG. 9 shows calculations of theoretical approximations for 530 nm (Green) LED.

FIG. 10 shows spectral response calculations. Left: scaling and leakage current adjustment at 515 nm LED. Right: spectral response of no medium.

FIG. 11 shows temperature and fluid experiment results for the devices described herein. Left: results form the temperature experiment. Right: results from the flid experiment.

FIG. 12 shows intensity calibration at each Kp for 530 nm LED of the devices described herein.

FIG. 13 shows spectrometer comparison experiment results. Left: comparison results with a benchtop spectrometer. Right: the experiment setup.

FIG. 14 shows comparison of experimental measurements and theoretical approximation for each LED.

FIG. 15 shows spectral response for mediums.

FIG. 16 shows glucose device design and initial prototype. Left: a glucose form factor design. Right: a glucose monitor prototype.

FIG. 17 shows a device for monitoring glucose according to the present disclosure.

FIG. 18 shows a device prototype. Left: GlucoLux finger scan prototype. Right: GlucoLux multi-spectrum reading.

FIG. 19 shows a block diagram of GlucoLux Device single PCB. Top-side Showing the surface mount LEDs, and the PDs. Bottom-side showing the microcontroller, LED driver, and power supply.

FIG. 20 shows a data processing pipeline for the processes described herein.

FIG. 21 shows a graph of glucose level distribution.

FIG. 22 shows skin tone effects on spectral signal and feature generation for normalization.

FIG. 23 shows Clarke error grid analyses. Left: 5-fold CV. Middle: LOO CV with calibration. Right: LOO CV without calibration.

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” can include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

### GlucoScan Device

Diabetes is a disease that impacts around 400 million people across the world. It causes numerous issues including shortened life expectancy, decreased quality of life, cardiovascular issues, and more. Prediabetes can be a precursor to diabetes characterized by blood sugar levels that are higher than normal but lower than diabetes. Prediabetes may be treatable and reversed through lifestyle changes, but most people are unaware they have it. Diabetes is traditionally monitored with frequent blood glucose testing which is invasive and requires drawing blood for accuracy. By tracking blood glucose and overall trends, we can monitor the progression of prediabetes. Blood glucose monitoring has other applications outside of diabetes and has led to a new class of devices that are less invasive by tracking biomarkers as estimations of blood glucose. Continuous Glucose Monitors (CGM) measure interstitial fluid in the body to provide frequent glucose readings. Breath Ketone Analyzers measure the ketones in your breath. These devices have the ability to less invasively monitor proxies for blood sugar levels. Knowing your personal blood glucose response can help people make blood sugar conscious food choices and identify precursors to diabetes.

Thus, a noninvasive continuous blood glucose monitor can lead to advancements in diabetes monitoring, prediabetes identification, and nutrition planning. The goal of this study is to demonstrate a proof of concept for such a device. Described herein is GlucoScan, a non-invasive system that uses optical spectroscopy to monitor change in blood glucose. This not only allows us to take instantaneous measurement but is capable of generating longitudinal datasets. The results of the study show a strong correlation of various wavelengths in the visible region with blood glucose. It should be understood that the GlucoScan device is used to illustrate the disclosed technology and that the disclosed technology is not limited to the technology as implemented in GlucoScan.

### Device

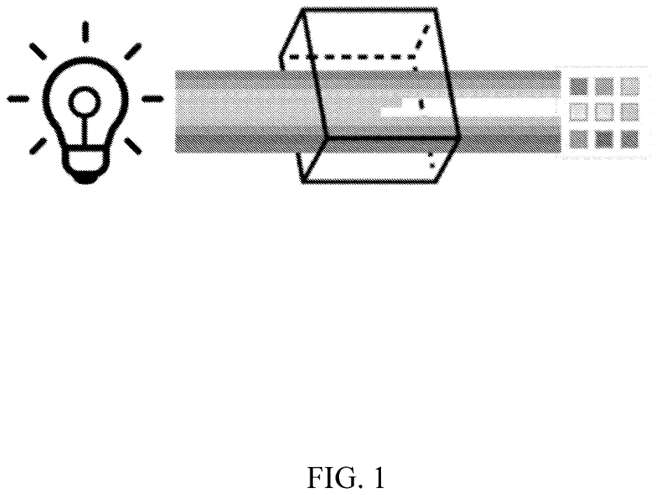

Optical spectroscopy is the interaction between optical photons and matter. Given a light source, a medium, and a set of photodetectors, as shown in FIG. 1, we can measure the optical response of the medium. When photons are absorbed by the medium, there will be fewer photons measured by the photodetectors. When photons pass through or excite the medium, there will be more photons measured by the photodetector. To create a wearable optical spectrometer, there are two choices: reflection and transmission. Reflection is commercially available in devices like smartwatches, while transmission appears in devices such as fingertip pulse oximeters. As an illustration, we decided to focus on transmission sensors to reduce parameter consideration, such as angle of the source, and literature supports the decision for transmission measurements. Thus, we use transmission fingertip spectroscopy in a similar design to a pulse oximeter.

Glucose measurement via blood sample is the gold standard. It has been tested, validated, and proven effective in diabetes management. Due to the invasiveness of drawing blood, research has begun into less invasive optical methods. These works have shown promise for measuring glucose in the visible and near infrared range. Specifically, shows the fluorescence emission of glucose oxidase has a peak at around 520 nm and increases in the presence of glucose. Work from found that as glucose concentration increases, the ratio of the 510 nm wavelength to 475 nm wavelength of transmission decreases, using Beer-Lambert's law. Additionally, 650 nm wavelength of light impinging on a solution has been shown to increase, in transmittance of photons, with glucose. Therefore, in this study, we focused on three wavelengths: 475 nm, 515 nm, and 680 nm. For each of these wavelengths we chose a narrow band LED and photodetector centered at the wavelength.

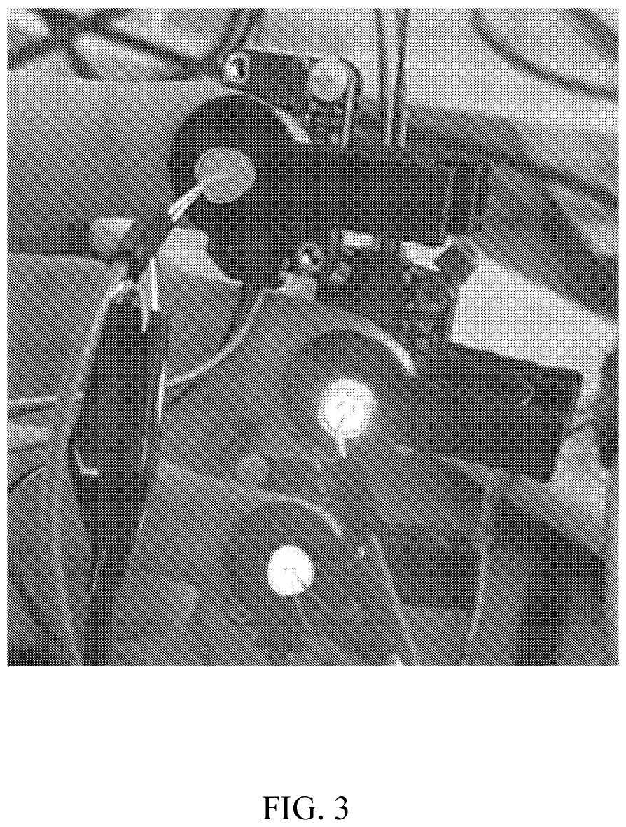

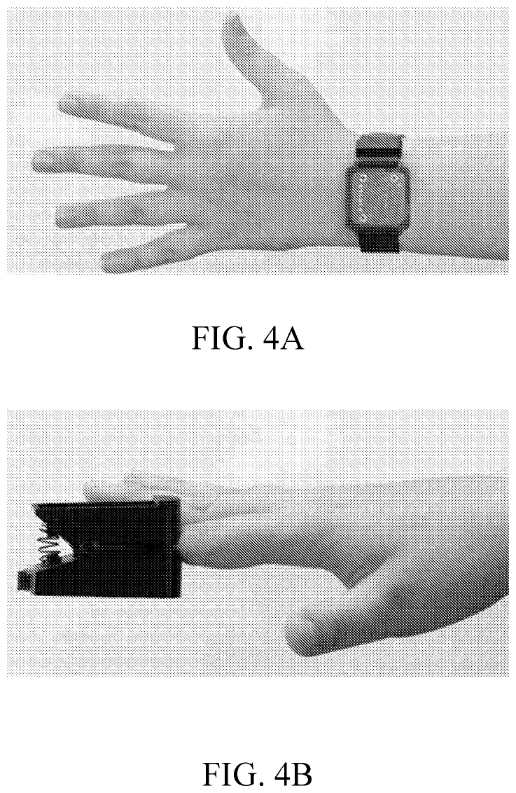

The device described above is shown in FIG. 3. Its components are as follows: The AS7341 spectral sensor with photodiode array that senses between peaks of 415 nm to 680 nm in the visible range. The device has photodiodes at 480 nm, 515 nm, and 680 nm. An Arduino Nano 33 IoT to record the data and send it to our data collection system, Raproto. The three LEDs at 470 nm, 515 nm, and 680 nm wavelengths. A 3D printed form factor was used to house the components and snugly attach to the fingertip.

### Study and Results

We conducted a preliminary user study on a single participant where we tested the disclosed system compared to a commercially available glucometer. We attached the GlucoScan devices on the participants left hand as shown in FIG. 3. The 680 nm LED was attached to the index finger, the 515 nm LED was attached to the middle finger, and the 475 nm LED was attached to the ring finger. This setup is shown in FIG. 3. Blood glucose readings were drawn from the right hand. This study was performed over approximately half an hour. Our participant started in a fasted state which is shown with the low starting glucose readings. At the start of the study, they drank a sugary drink, and we recorded the effect on their blood sugar. Approximately every two minutes, we took a new blood glucose reading. We continuously sampled the three GlucoScan devices at a rate of approximately 2 Hz.

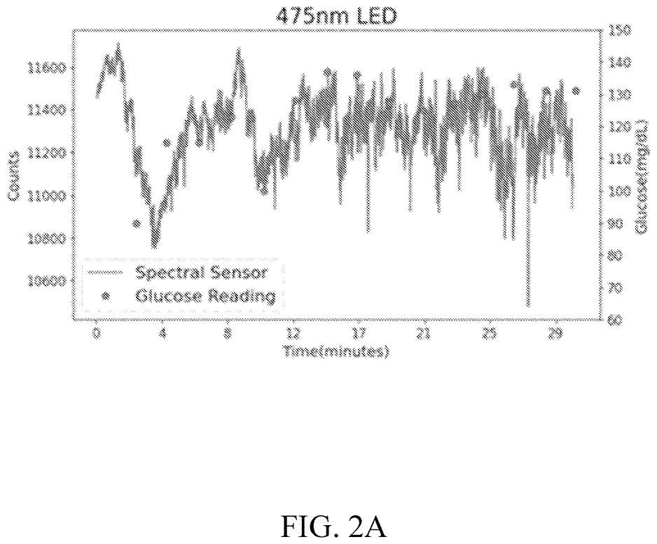

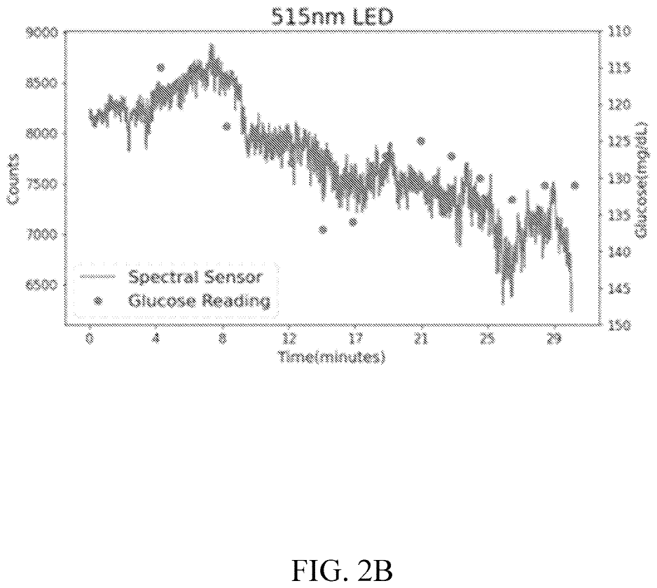

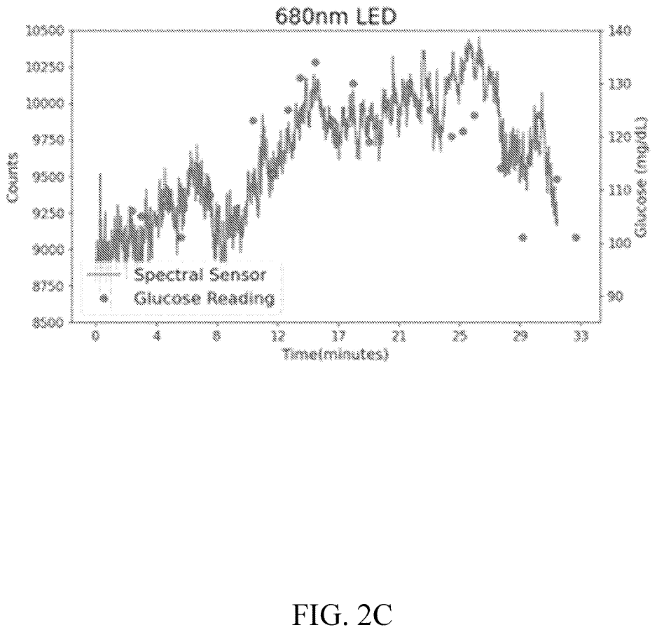

The results of the study are presented in FIG. 2. The patient's blood glucose, as measured by a commercially available glucometer, is shown by the red points and the optical spectroscopy readings from the 480 nm, 515 nm, and 680 nm photodetector channels are shown in blue, respectively. The glucose readings were manually calibrated when overlaying the data. The maximum photodiode counts are 216, and the initial intensity of the transmitted response was calibrated to be relatively strong. In FIG. 2A and FIG. 2C, the change in counts positively trends with the change in glucose. In FIG. 2B, the change in counts negatively trends with the glucose readings, and the y-axis of the glucose readings is flipped. After interpolation and detrending of the signals in FIG. 2, 470 nm and 515 nm are shown to be statistically significant with p-values of 0.344 and 0.6. These results show that readings from the GlucoScan sensor may be used to track overarching glucose trends.

### Conclusion

This study expands on research that blood glucose can be measured via optical spectroscopy. The results show that blood glucose increases with light transmittance from a 475 nm LED and inversely with that of a 515 nm LED.

### Lumos Device

Spectroscopy, the study of the interaction between electromagnetic radiation and matter, is a vital technique in many disciplines. In medicine, spectroscopy has facilitated substantial advancements in diagnostic technology. Developments in spectroscopy have brought about biomedical imaging technology, including X-ray, MR spectroscopy, and CT scan technology. Additionally, analysis of biological samples relies heavily on spectroscopic techniques to measure the biomarkers that track the progression of diseases. In its current instantiation, its utility is limited because it only provides a brief snapshot of the monitored parameter. Data in isolation, not in a time series, does not provide the trends clinicians rely upon to make informed decisions.

Wearable technology brings sensing and tracking technologies to the masses. Integrating this technology into our daily lives has collected a wealth of previously untapped data. Capturing this amount of data has created longitudinal datasets ripe for analysis. These datasets present opportunities for the application of machine learning and data analytic techniques. These data, combined with machine learning and artificial intelligence, can support clinical decisions and provide more accurate diagnoses. More so, the wealth of data has transformed how people live, impacting their daily routines, interactions with others, and health monitoring. These qualities make wearable technology the perfect vehicle to collect continuous data from spectroscopy devices.

The traditional benchtop spectrometer is already being miniaturized. Sampling and testing are being done in the lab, clinic, and field via portable spectrometers. Optical sensing has demonstrated the viability of a wearable spectral device but is limited by its sensing spectrum. Providing researchers, clinicians, scientists, and even the general public with a more comprehensive wearable spectrometer will increase the amount of meaningful data collected as well as lead to novel clinical applications. Compared to prior work, the disclosed approach creates a ready-to-use research platform that can be utilized to develop these novel applications and longitudinal datasets.

In this disclosure, we explore the following:
How can we redesign the traditional benchtop spectrometer while accounting for the many constraints of a wearable device including battery life, computing constraints, and overall footprint?
How can we determine the spectral response of a medium while adapting to dynamic environments given a limited set of light emitting diodes and photodiodes?
How can this device be leveraged to address research challenges in target applications?

The potential benefits of integrating spectroscopy into wearable technology are substantial. In this work, we present Lumos, a wearable spectroscopy device that enables noninvasive, real-time, and continuous health monitoring. To make Lumos available to the research community, we open-sourced the hardware designs and algorithms used in this work. This includes the components, schematics for the circuitry, Gerber files for printing the PCB, and code. Lumos uses off-the-shelf components that can be combined in the manner described in this paper for research purposes or be tailored to fit a targeted application. We evaluated our device on its accuracy when compared with a traditional spectrometer, its power consumption, and its reactions to temperature and fluid. It should be understood that the Lumos device is used to illustrate the disclosed technology and that the disclosed technology is not limited to the technology as implemented in Lumos.

We developed an algorithm to detect the spectral response of a medium. This determines the optimal spectrum for further evaluation when creating a wearable spectroscopy device for a target application. This algorithm seeks to construct the spectral response of only the medium. It adapts to light leakage from the environment providing maximal invariance to environmental disturbances. It adjusts its light intensity to standardize readings across skin tone, change in pressure applied to the device, blood perfusion, etc. Additionally, we estimate the theoretical response of our device without a medium to remove the bias from our readings. We evaluate this algorithm on its accuracy, resolution, and resistance to outside sources of noise.

Lumos is an open source device that facilitates wearable spectroscopy research in target applications, as shown by the described evaluation. In this evaluation, we used the Lumos device in a medical application: prediabetes monitoring. First, we monitored blood glucose via a glucometer and compared it to readings from our device. We used the spectral response estimation algorithm to determine the optimal wavelengths for tracking glucose. Then we created a customized device tailored to those wavelengths and demonstrated that the device could be used to track changes in glucose. This can track the progression of prediabetes for our target application.

As explained herein, this disclosure provides as least the following:

An open-source device to perform wearable optical spectroscopy research. This device is designed to get researchers up and running quickly in new studies and be easily tailored for custom applications.

An algorithm to estimate the spectral response of a medium to determine the optimal spectrum for a given application while providing maximal invariance to environmental disturbances.

A pilot study, prediabetes monitoring, to demonstrate the viability of using Lumos for a target applications.

### Spectroscopy

Spectroscopy studies the interaction between electromagnetic radiation and matter. It is a vital technique in many disciplines, including medical imaging, molecular analysis, and remote astronomical sensing. The most common types of spectroscopy include atomic spectroscopy, ultraviolet and visible spectroscopy, infrared spectroscopy, Raman spectroscopy, and nuclear magnetic resonance. Traditionally, spectrometry, the measurement of the interaction between electromagnetic radiation and matter, is done on benchtop requiring large, heavy, and complex machines with operators that have been specially trained. These benchtop services are characterized by the precision and accuracy of their measurements, but due to the complicated processes, procedures can be time-consuming and inconvenient. Furthermore, these techniques cannot be directly applied to living organisms as normal physiologic functions must be maintained. Hence, samples are obtained from living organisms, but this is not an entirely benign process as it typically requires invasive procedures that cause subject discomfort.

Work has already begun to miniaturize the traditional benchtop spectrometer. There is still a need to track real-world, longitudinal continuous data on patients. For example, the most common issue with pain management protocols is the insufficient treatment of pain with opioids. The reasons for insufficient pain management can be caused by one or more of the following: intolerance of side effects from the medications, desire to minimize medication intake, cost of medication, and external sources of influence. Current techniques for monitoring adherence involve lengthy questionnaires and invasive testing. Whereas a wearable spectrometer could solve this adherence problem by continuously monitoring patient opioid use. Additionally, it could help identify drug diversion, a major concern for clinicians and the Drug Enforcement Agency (DEA). Further, opioid management during in-patient hospice care is an exceptional clinical challenge. The goal is to provide sufficient analgesia for conditions that can be particularly painful. Balanced against this is the desire for a patient to maintain normal cognitive function. Additionally, during hospice care, the goal is to provide the best quality care while minimizing invasive techniques. Rather than drawing blood samples to monitor a patient's opioid levels, a non-invasive monitor would greatly benefit both the patient and clinician. In addition to the above examples, alcohol, ketones, blood pH, glucose, as well as responsiveness to medical treatment and medications could all benefit from being monitored in a continuous, non-invasive, outpatient manner.

### Applications of Wearable Spectroscopy

In research, many forms of spectroscopy have been explored for wearable sensing. Most importantly, near-infrared and functional near-infrared spectroscopy has been utilized for physiologic measurements. Near-infrared spectroscopy (NIR) utilizes the wavelength range from 750-2500 nm of the electromagnetic spectrum and has been commonly used to measure oxygenated and deoxygenated hemoglobin through the skin. Functional near-infrared spectroscopy (fNIRS) uses a subset of the NIR spectrum to sense the oxygenation of hemoglobin. Diffuse optical technologies is a model-based technique for NIR measurement done via continuous-wave, frequency-domain, and time-domain spectroscopy. This technique has been used to measure the concentrations and change of hemoglobin oxygenation, water, and lipids. Here, we will present some applications that are becoming possible due to wearable spectroscopy.

Non-invasive blood sugar monitoring has been an elusive objective. Historically, a blood sample is required to detect the concentration of glucose. This is a painful process that creates a barrier to consistent and effective blood sugar monitoring. NIR spectroscopy, a non-invasive modality, has shown potential for detecting glucose. To date, attempts are complicated and plagued by obstacles such as interfering absorption and motion artifacts. Through our research, we have demonstrated the ability to noninvasively and continuously monitor changes in glucose in limited circumstances.

An increased concentration of lactate indicates insufficient blood flow or excessive metabolism. This can have many causes and even more numerous complications. One example is the end organ damage caused by sepsis. Traditional spectroscopy is excellent at detecting the concentration of lactate in a blood sample. Unfortunately, ascertaining the clinical relevance of a single data point can be challenging as it lacks context. Research into a wearable lactate sensor has shown that the NIR region holds promise for detecting changes in lactate concentrations.

The most studied wearable spectroscopy devices use functional near-infrared spectroscopy to detect brain activity. When a region of the brain is more metabolically active, it has increased oxygen requirements. This is detected and provides use in brain-computer interfaces. Diffuse correlation spectroscopy and speckle contrast spectroscopy are applications of functional near-infrared spectroscopy that have been used to measure deep tissue blood flow. Continuous wave diffuse optical imaging implemented in a wearable device has allowed for the measurement of oxygenation changes in breast tumors during chemotherapy. This example, non-limiting device uses six LED and photodiode pairs, and can provide clinicians with the ability to assess the effectiveness of their treatment and modify it in real-time.

Wearable technology provides the ability to simultaneously attach sensors to many areas of the body. This allows for the capture of a diverse set of physiologic characteristics in real-time. Further, it promotes research outside of the lab for situations in which it is unreasonable to use traditional benchtop methods. Spectroscopy is a powerful tool that is essential for analyzing and identifying human disorders. Thus far, it has been an underused modality limited to providing transient snapshots of data. Advances in wearable technology have allowed for the application of spectroscopy techniques to health sensing. Moving forward, wearable spectroscopy provides the opportunity to continuously track the progression of disease or even health of a subject. It creates an opportunity to collect massive amounts of high-value longitudinal data, which data can be used to gain insights into human health and performance.

### Theory of Operation

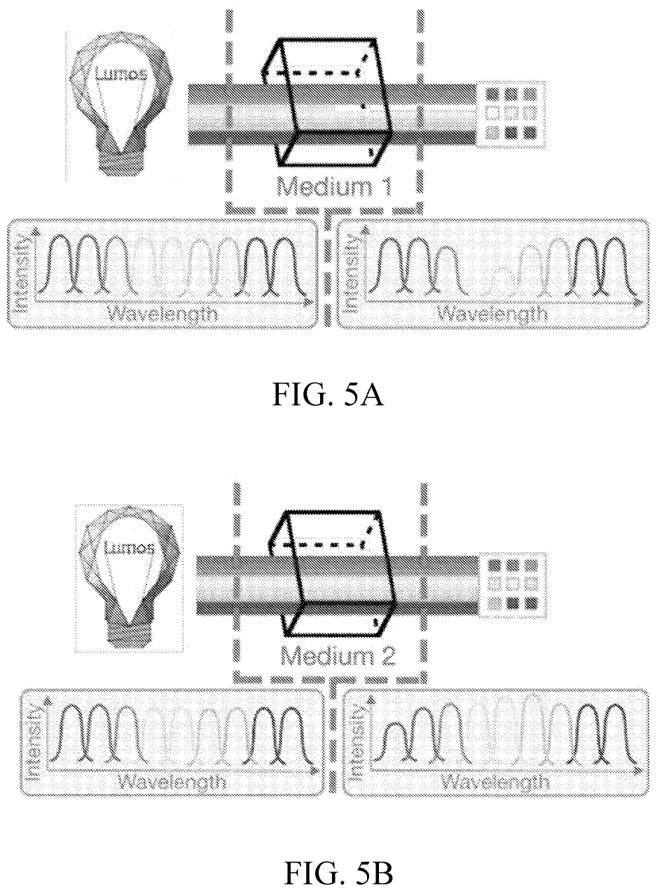

Spectroscopy, as previously stated, is the study of the interaction between electromagnetic radiation and matter. It has two main interactions: absorption and emission of photons, particles of light. The measurement of the photons gives information about the medium. Absorption is when electromagnetic radiation, such as light, is absorbed via a change in energy as it passes through a medium. The wavelengths that are absorbed or partially absorbed would present a lower number of photons at the detector, while the wavelengths that were transmitted would present a higher number of photons. This is depicted in FIG. 5A. The figure displays a multicolored light shining on and interacting with a medium. That medium absorbs a portion of the spectrum, yellow wavelengths. The remaining light is read by the sensor on the opposite side of the medium, showing lower counts at the sensors near the yellow wavelengths.

Emission is when the electromagnetic radiation applied to a medium results in the release of photons due to absorption of photons that were applied. This is depicted in FIG. 5B. The figure displays a multicolored light with various center wavelengths shining on and interacting with a medium. This medium emits photons which are visible to the human eye as green light when illuminated by the multicolored light. This release, as well as any transmitted light, is captured by the detector.

The emission of photons must obey the conservation of energy. Thus, the emission would not be greater than the energy absorbed. These measurements grant us insight into the medium itself. Mediums can have both absorption and emission occurring at once. Thus, we use this theory as a basis for our sensor and algorithm design in this paper.

Traditionally, optical spectrometers measure the photons either transmitted through a medium or reflected from a medium. Transmission-based spectrometers place the sensor on the opposite side of the medium from the light source. Reflectance-based spectrometers place the sensor and light source on the same side of the medium. Each method has its advantages and disadvantages. To discuss the differences between the two in a wearable configuration, we will use pulse oximetry as an example. Pulse oximetry is a non-invasive application of spectroscopy to monitor oxygen saturation. Studies have shown both transmission and reflection-based devices can provide accurate oxygen saturation measurements. Transmission-based devices, such as the finger clamp commonly used in medicine, are best used in thin areas where the entirety of the medium can be perfused with light. While this limits the on-body locations in which the sensor can be placed and the wearers' movement, this style of sensor is trusted in medical settings. Reflection-based pulse oximetry is generally housed in a smartwatch. It is useful when light cannot fully travel through the medium to show an optical response on the other side. The light source and sensor are placed side by side for reflection-based measurements, which gives more flexibility for on-body locations of measurement. However, light source and sensor alignment are more complicated with this method. As such, a small change in the angle of a surface mount light emitting diode (LED) can drastically change a measurement. As both methods have pros and cons, we seek to incorporate both into our designs. This can require additional data processing as well as separate form factors.

When designing Lumos, we pursued designs that would measure absorption and emission as well as measure photons through transmission and reflectance.

### Form Factors

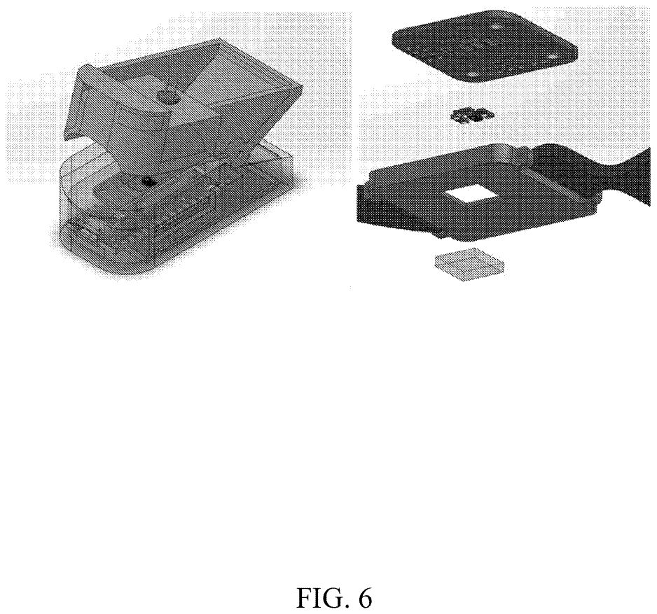

As we developed Lumos, we pursued two example form factors, a wristband form factor with a similar design to a smartwatch and a finger clamp form factor with a similar design to a pulse oximeter. These designs are shown in FIG. 6. The finger clamp leverages transmission spectroscopy and places the light source on the opposite side of the medium from the detector. The wristband leverages reflectance spectroscopy by placing the light source and photodetector on the same side. Both the wristband and finger clamp allow for emission and absorption spectroscopy based on the spectrum input. These form factors offer users a more comfortable experience when using our devices. Furthermore, as these form factors are mainly 3D printed, with a few additional easy-to-find components, they do not require a large time or resource investment.

### Finger Clamp

The first form factor is the finger clamp, and it is shown in FIG. 6. It is similar to commercially available pulse oximeters in that it rests on the distal phalanges of the finger. This form factor was chosen because it is comfortable, easy to use, and generally familiar to people. It leverages transmission spectroscopy in which light shines on one side of a medium, with the detector on the other side reading the photons from absorption or emission. The finger clamp consists of the 3D printed housing, a spring to keep the housing closed, and a bevel pin to open and close around.

When designing this form factor, we addressed two main challenges: consistent pressure from the clamp without causing discomfort and light leakage from the environment. Consistent pressure is needed because as pressure varies, the measurements also fluctuate. So, we introduced a spring and pin to standardize the amount of pressure applied while remaining comfortable to the wearer. Light leakage is the environmental light sensed by the detector. If too much light leaks in, it can obscure subtle changes in our measurements. To address this, we closed off the sides of our form factor as shown in FIG. 6. Additionally, through hole or surface mount LEDs can be used in this form factor.

### Wristband

The second form factor is a wristband, as shown in FIG. 6. This is similar to a smartwatch in how it rests on the wrist. It has the advantages of being familiar to the user, comfortable, and can be worn all day. The wristband form factor is based on reflectance spectrometry, so the light source and detector are on the same side of the medium. Because of this, we are less limited in the locations this sensor can be placed. For example, if we extend the straps, it could be attached to the upper arm or leg. This form factor consists of the 3D printed housing, straps, and a skin-safe encapsulant. The skin-safe encapsulant separates the electronic components from the skin, which is essential in a wearable device. It is a transparent silicon that provides a moisture barrier from any sweat on the skin. The entire assembly protrudes from the bottom of the wrist band to ensure tight contact with the skin. Additionally, this encapsulant allows for the device to be worn tightly on the skin, decreasing the amount of light leakage and standardizing the amount of pressure applied.

### Components

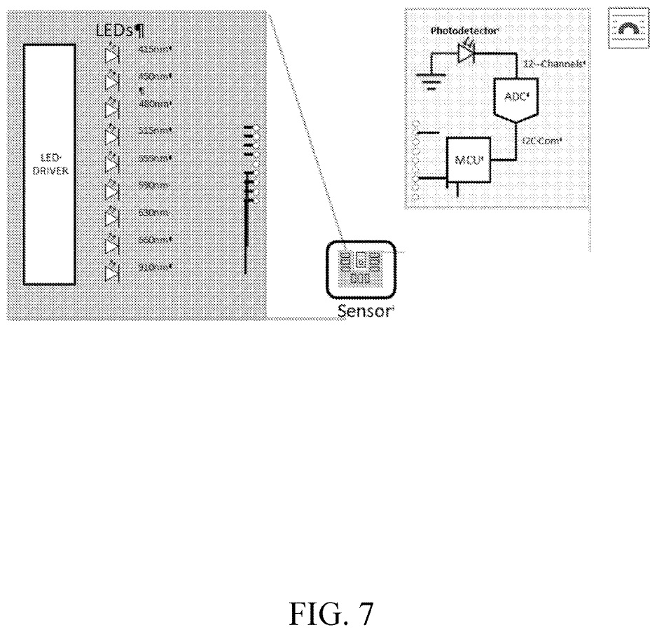

Both form factors described above are based on the same electrical components; they are housed in different configurations. At a high level, the Lumos device consists of a light source and a detector. The light source we describe includes commercially available surface mount LEDs that cover the entire visual spectrum and LED drivers to control the intensity of the light. The LEDs are configurable based on the spectral needs of the application. The detector consists of the sensor with the spectral sensor, microcontroller, communication component, and battery. To house these electronics, we developed a small custom printed circuit board (30 mm×30 mm×1.6 mm) to ensure a small footprint to fit into our form factors. FIG. 7 shows the component diagram for our device.

### Light Source

The goal of the light source is to cover the targeted spectrum and appropriately illuminate the medium. This is accomplished through an array of surface mount LEDs and LED drivers. In the finger clamp, the light source sits on top of the form factor. In the smartwatch, it is integrated into the same printed circuit board (PCB) as the photodetectors.

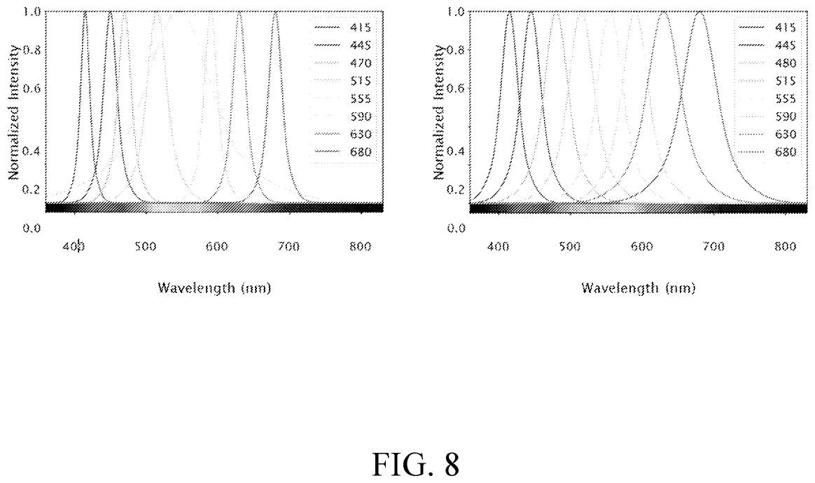

To build a comprehensive system, in this paper, we selected an LED array that covered the visual spectrum. Each LED produces light in a continuous spectrum centered around one peak wavelength. We have eight LEDs in the visual spectrum with center wavelengths around 415 nm, 450 nm, 470 nm, 530 nm, 568 nm, 599 nm, 633 nm, and 660 nm. The LEDs do have spectral ranges that overlap. This is shown in FIG. 8. In this figure, the total spectrum covered is shown as well as the overlap between LEDs. To handle the complexity that the overlaps may cause, we developed an algorithm discussed herein.

To appropriately perfuse the medium, we use LED drivers to tailor the intensity of each LED. This is important so that the intensity of the LED is high enough for a meaningful measurement but not too high that it will oversaturate the photodiode. The LED drivers also allow us to have consistent readings between all LEDs.

### Detector

The detector senses the response from the light source applied to the medium. It consists of the spectral sensor, microcontroller, and communication component. The spectral sensor is integrated into our custom PCB. The microcontroller and communication component are housed on a separate board.

We chose the A7341 spectral sensor because it has a large sensing range and can easily be integrated into a wearable device. It has eight channels in the visible range, with peaks centered around 415 nm, 445 nm, 480 nm, 515 nm, 555 nm, 590 nm, 630 nm, and 680 nm. The sensing channels are displayed in FIG. 8. The dimensions of this device are 3.1 mm×2 mm×1 mm. Thus, it fits into our form factors nicely, as shown in FIG. 8. The AS7341 needs 1.8 VDD for operation. The max current draw from the AS7341 is 300 μA making it low power.

We chose the Arduino Nano 33 IoT to power our AS7341 and communicate data from the AS7341 to the data collection system. The Arduino Nano 33 IoT has a Cortex M0+ SAMD21 processor and NINA-W102-00B WiFi/BLE Module. It has the ability to send data out over WiFi using MQTT to the data collection platform as it eliminated our need for an additional device such as a smartphone to route the data to the data collection system. The Arduino and the entire device are powered by a 400 mAh lithium-ion battery. The Arduino supplies 3.3V, which is more than needed for our spectral sensor. As such, there is a voltage regulator on the AS7341 so the Arduino can safely power the spectral sensor.

### Intensity Calibration

LED light intensity is affected by many factors, including environmental light, skin tone, change in pressure applied against the device, and blood perfusion. To remove the impact of these factors, each LED's intensity is calibrated for each person and environment. This one-time calibration process occurs when the user first puts it on. It takes approximately 2-5 seconds to calibrate each time. The calibration process is necessary to mitigate over and under saturation on the detector side. Overall, the calibration process adjusts the light intensity until a targeted reading is obtained at the photodetector. We calibrate each LED's brightness on the photodetector nearest to its wavelength. For example, the 470 nm LED will be calibrated with the 480 nm PD.

Our photodetectors have a 16-bit resolution giving us a maximum value of 65,535. To leave room to sense both absorption and emission, we choose a target value of approximately 2 of our maximum reading or between 42,000 counts and 44,000 counts. This gives our sensor a higher sensitivity while leaving ample room for emission readings. This can be adjusted based on the needs of the application. The iterative calibration process is performed by adjusting the current to the LED using an 8-bit LED driver that adjusts the light intensity until it reaches our targeted value. This adjusts the LED with a resolution of 0-255, approximately 0-50 mA. At each iteration (i), we calculate the error between the targeted reading (rₜ) and the current reading (rᵢ). A proportional controller is then used to determine the adjustment required for each LED to achieve the desired count value. A proportional gain (Kₚ) is used to determine the ratio of output response to the error signal. At each iteration, we calculate the intensity (I) with the following equation:

$${I}_{\text{?}} = {K}_{p} ( {r}_{t} - {r}_{i} ) \qquad ( 1 )$$

$$\text{?} \text{indicates text missing or illegible when filed}$$

Then after the new intensity is calculated, we adjust the LED and repeat the process until the targeted reading is reached. In general, the entirety of this iterative process occurs in approximately 2-15 iterations or 3-5 seconds.

### Environmental Light Leakage

The intensity calibration accounts for the initial environmental light leakage, but as environments change, so does the light leakage. To handle this dynamically, we sample our sensor in two LED states: ON and OFF. When the LED is ON, we read the combination of the spectral response of the medium and the environmental light leakage. When the LED is OFF, we only read the environmental light leakage. The time spent in an ON state is determined by each LED's rise time and the integration time of each of our PDs. The rise time is the time it takes for the LED to reach maximum intensity based on the current calculated in the intensity calibration. The integration time is the amount required for each PD to record a reading. The rise time and integration time are gathered from the datasheets. Then, to eliminate the environmental light leakage from our reading, we take the average of the two OFF states that surround an ON state and subtract that from the ON state. This is done for every PD for each LED.

### Gaussian Estimation

Theoretically, the spectral response of our LEDs and PDs are approximately a Gaussian. To estimate the Gaussian, we need three metrics: intensity, center wavelength, and full width half maximum. The intensity (I) is gathered from the intensity calibration step described above. The Gaussian estimation will be scaled to the calibrated intensity. The center wavelength and full width half maximum are available from the datasheets accompanying each LED and PD. The center wavelength (CW), is the wavelength at which the LED or PD is at maximum intensity. The full width half maximum (FW HM), is the width of the spectrum at half of the full intensity. When calculating the Gaussian, the FW HM relates to the standard deviation σ by FW HM=2σ Sqrt(2 ln 2). Then, we estimate the spectral response (SR) via the following equation:

$$SR = I e exp - {( x - CW )}^{2} / ( 2 ( FW HM / {2}^{2.35} ) ) \qquad ( 2 )$$

The estimated curves for all LEDs are shown in FIG. 8, left, and for all PDs are shown in FIG. 8, right.

### Spectral Response Calculation

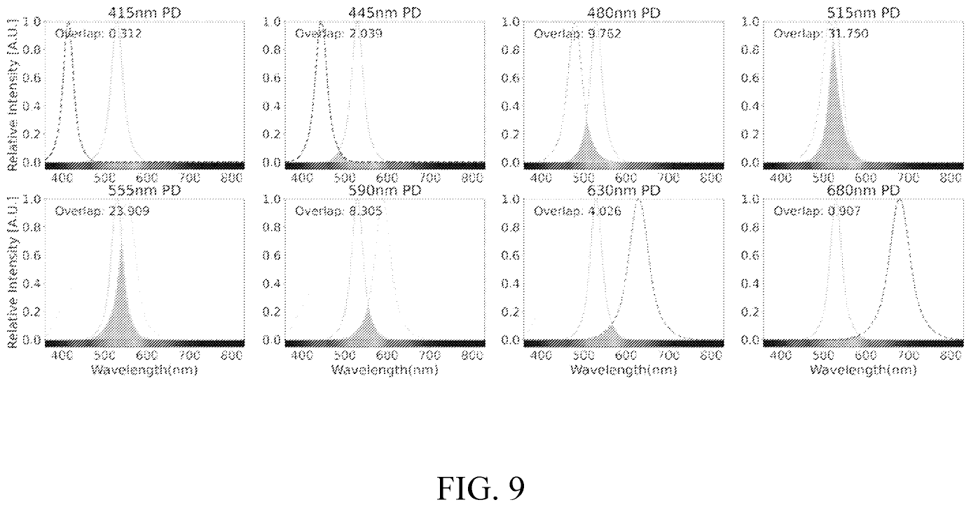

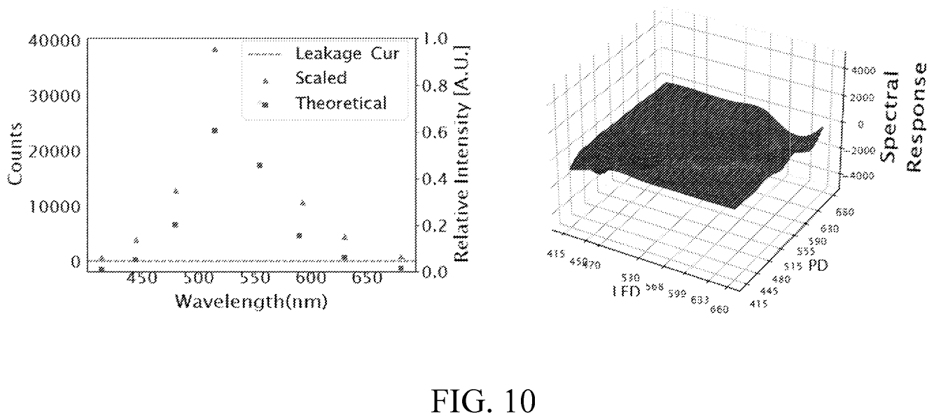

Next, we calculate the expected response for each combination of LED and PD. To do this, for each LED, we overlay each PD. In FIG. 9, we show this example for the 515 nm LED. This LED is shown with each PD, giving us eight graphs. When the LED and PD overlap, we expect a spectral response from the Lumos device. In FIG. 9, we see that the LED overlaps with the 445 nm, 480 nm, 515 nm, 555 nm, 590 nm, and 630 nm PDs. For each overlap, we calculate the area under the overlap of the two curves to determine the expected response of each PD. This is normalized to the total possible response from each PD, i.e., the area under the PD curve. We show the estimated responses for the 515 nm LED in FIG. 10, left. Then, we scale the estimated response to the counts we read from the Lumos device, also shown in FIG. 10, left. In total, for the eight LEDs and eight PDs, we calculated 64 expected responses.

### Leakage Current Adjustment

Photodetectors are not perfect and a known issue is that they produce a leakage current when any photons excite the photodetector. This causes small readings in channels that are not being excited. The readings are linearly related to the number of photons hitting the sensor, i.e., as more photons hit the sensor, the higher the readings. We take an experimental reading without a medium to calculate the leakage current adjustment. We look to the PD readings where the estimated response is zero and average them. This average becomes our current leakage adjustment which is added to all theoretical estimations where the estimated response is zero. This is shown in FIG. 10.

### Overall Spectral Response

Finally, we calculate the spectral response of the medium by combining all the PD readings from all of the LEDs. We show the overall response of air or no medium in FIG. 10, right. The overall response is represented by the entire surface in the figure. The peaks and troughs represent wavelengths with high responses that should be targeted for higher resolution sensing. Peaks represent absorption wavelengths, while troughs represent emission wavelengths. In this figure, we see very little response, which is expected as it is the spectral response of air.

### Temperature Experiment

Temperature can affect sensor readings even when not outside of the normal operating range. To under-stand the effect of temperature on our device, we evaluated the sensor's response to hot and cold temperatures. The sensor, AS7341, is guaranteed to work within the range 30° C. to 70° C. Since we do not expect our sensor to experience the extremes of this range when worn on the body, we evaluate our device between 0° C. and 45° C. These temperatures were selected as they are on the edges of normal environmental conditions. For any wearable device, it is important that it withstand normal environmental conditions that humans experience.

To evaluate our device in varying temperatures, we chose a narrow spectrum light source (445 nm) that would illuminate a single channel. We tracked the counts on that channel as we varied the temperature between 0° C. and 45° C. This experiment was performed with our sensor in a black box to minimize ambient light. To increase the temperature, we used a hair dryer to blow hot air through a hole in the side of the box while not increasing the ambient light. To decrease the temperature, we put the black box into a freezer. In both scenarios, we tracked the temperature with a thermometer. Furthermore, we repeated this experiment across multiple devices to show inter-device repeatability.

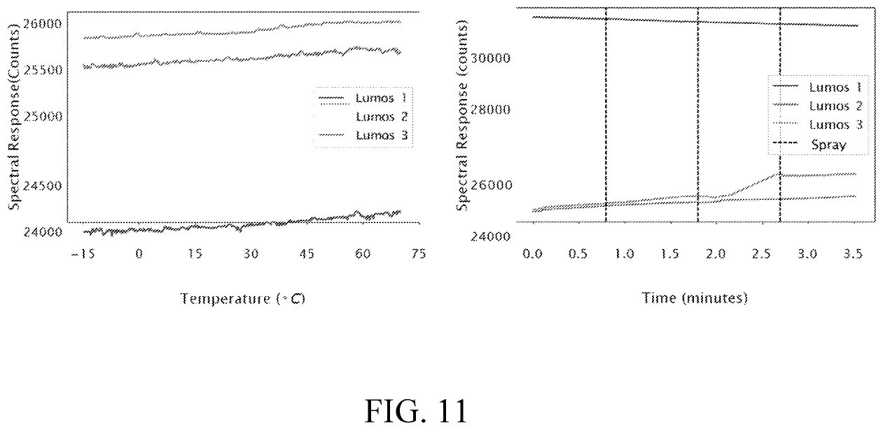

We display the results from the experiment in FIG. 11, left. We show the main channel (445 nm) from three devices. The other channels had low counts that did not have noticeable changes related to temperature. The three devices all read the same LED but, due to their placement, received different numbers of photons. This accounts for the difference in counts between the devices. Over the 90° C. temperature change, we saw a less than 200 count change in our readings on any of the devices. This accounts for a less than one percent change in the overall reading.

### Fluid Experiment

In addition to temperature, fluids can also impact the sensors. For example, sweat can coat the encapsulant, causing differences in sensor readings. To evaluate Lumos's response, we simulated different levels of sweat. We used our wrist band form factor with a waterproof encapsulant to do this. This was done by spraying the encapsulant with water. We repeated this three times to simulate minimally sweaty to very sweaty, with very sweaty being someone dripping in sweat. Each spray was approximately 0.75 ml. As above, we performed this study in a black box to reduce light leakage. FIG. 11, right, shows the results from the fluid experiment in the 445 nm wavelength. All other wavelengths followed a similar trend. These results show no significant effect, less than 100 counts, on the spectral response readings until the third spray. This puts 2.25 ml of water onto our sensing area, well past what can be expected from sweating.

### Power

We measured the power consumption of the Lumos device using a digital multimeter to measure the power drawn when all LEDs are at max intensity, PDs are sampled at 10 Hz, and our communication protocols are functioning as normal. This is the max power draw from our device. With these settings, the Lumos device consumes 85 mA (425 mW) of current. This gives us approximately five hours of battery life with a 400 mAh lithium-ion battery. This is more than sufficient for a normal research study. Longer studies are possible with a higher capacity battery. Replacing this with a lighter-weight component can increase the battery life. In practice, our system could sample data less frequently or transmit data to the data collection system at higher intervals. A sleep functionality can also be implemented so that the device, including the LEDs, PDs, and processing circuitry, do not need to be on when not in use.

### Intensity Calibration

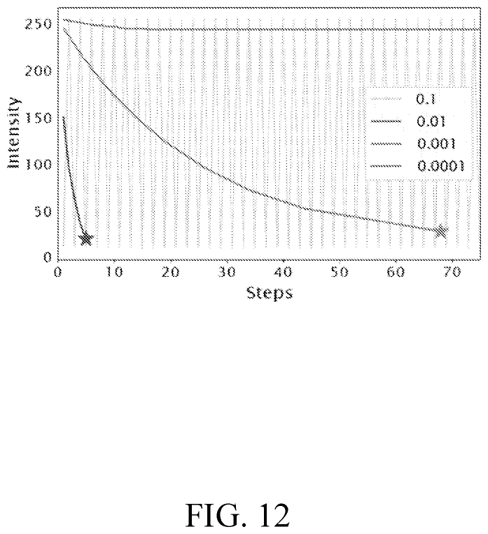

To determine the best Kp for our system, we tested four values, Kp=0.1, 0.01, 0.001, 0.0001. An initial default intensity value of/=255, maximum intensity, was used to start this test. We show the results for how I changed using the 530 nm LED at each step for each Kp in FIG. 12. The stars show when the calibration algorithm is successfully finished. Kp=0.1, 0.0001 did not converge to our targeted value even after running for over 100 iterations. Kp=0.01 converged the fastest with an average of 4.8 steps. Kp=0.001 converged in approximately 44 steps. We chose the setting Kp=0.01 for our device as it converged to the targeted value in the fewest number of steps. This took approximately 2.4 seconds. We show the results from each Kp in Table 1. This setting is adjustable based on the needs of the application. Anything above 0.1 or below 0.0001 does not converge, so we did not test those values.

TABLE 1
Time and Computational Needs for Each Kps

| Kₚ | # Steps | SD Steps | Time (s) |
|---|---|---|---|
| 0.1 | ∞ | ∞ | ∞ |
| 0.01 | 4.8 | 2.9 | 2.4 |
| 0.001 | 44.4 | 41.3 | 22.2 |
| 0.0001 | ∞ | ∞ | ∞ |

Comparison with Spectrometer

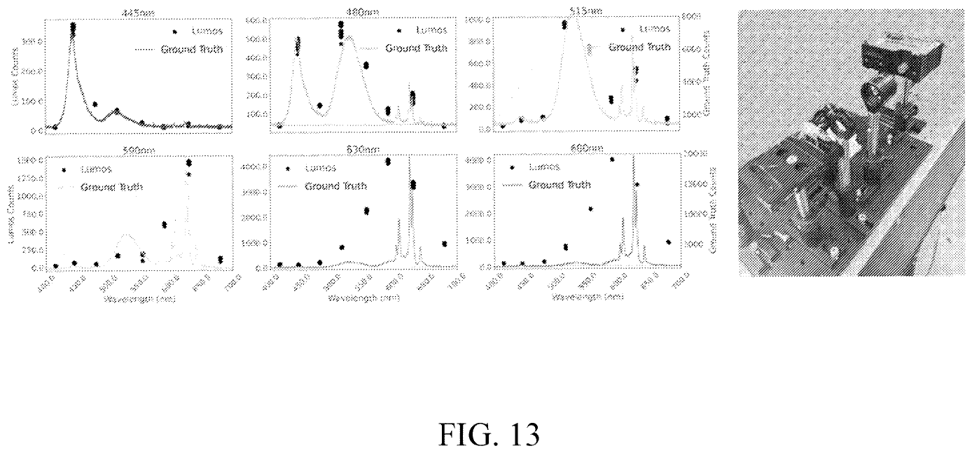

In order to validate the AS7341 spectral sensor, we compared it to a ground truth spectrometer, the Ocean Optics Vis-NIR fiber optic spectrometer, as seen in FIG. 13, right. This spectrometer has a range from approximately 450 nm to 1118 nm with a resolution of 1.37 nm. In this study, we generated colors that were similar to what our photodetectors read using an iPad screen. We pointed the iPad at both the ground truth spectrometer and the AS7341 and recorded the readings from both. In FIG. 13, left, the ground truth spectrometer readings are plotted alongside the AS7341 sensor readings. For the most part, we saw that the AS7341 readings followed the same curve as the ground truth spectrometer and gave confidence in moving forward with this device. We did see more error in the 630 nm and 680 nm readings. Without being bound to any particular theory, we believe the higher Lumos counts in the red spectrum were due to the PDs on our device having a larger sensing range in that spectrum.

### Theoretical Estimation

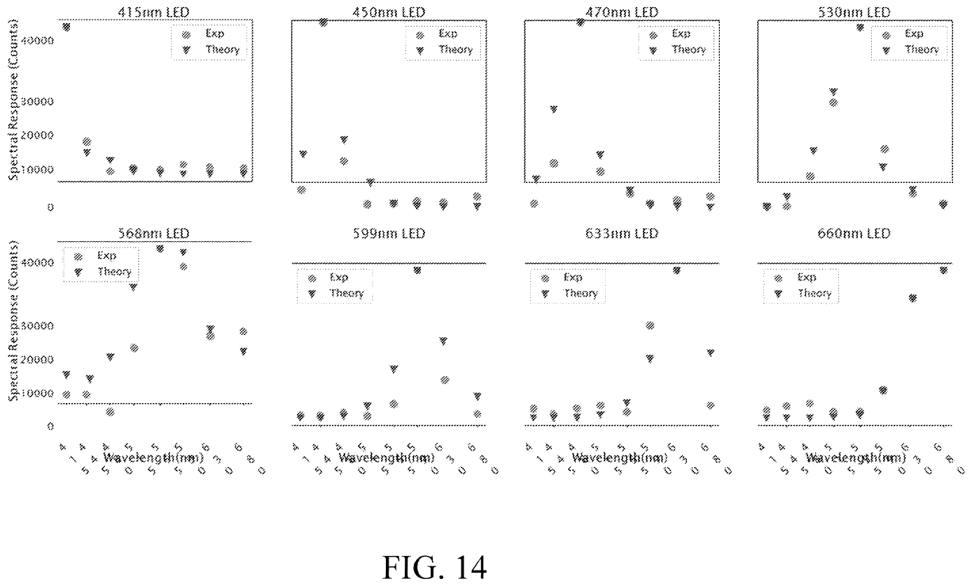

We evaluated our theoretical approximation by comparing them to experimental measurements. To record the experimental measurements, we placed the Lumos device in a black box. There were openings in the box for the wires with mitigations to remove all environmental light. We took measurements when the LED was off, and all channels showed zero counts. To have a consistent intensity across all of the LEDs, we used our intensity calibration algorithm to assure 44,000 counts. No medium was placed between the spectral sensor and LEDs. FIG. 14 shows the comparison between the theoretical approximations and experimental measurements at each LED. The theoretical approximations have a 0.944 Pearson correlation with the experimental measurements.

### Spectral Response of Medium

We conducted experiments to understand the Lumos device's ability to identify the spectral response of a medium. We started by characterizing the source and detector output with no medium and then moved on to characterize the change in output response when a medium was placed between the source and detector. We consider no medium to be air. The physical mediums we used were six colored light filters: purple, blue, green, yellow, orange, and red. The mediums were placed into the fingertip form factor for this experiment. In order to characterize these, we collected data to show the response of each of our LEDs when measured by all visible photodiode channels for each filter, including with no filter. The algorithm discussed in Section4 is applied to calculate the spectral response of the mediums or lack thereof. We compare this to the known spectral response of the mediums that we collected from the datasheets.

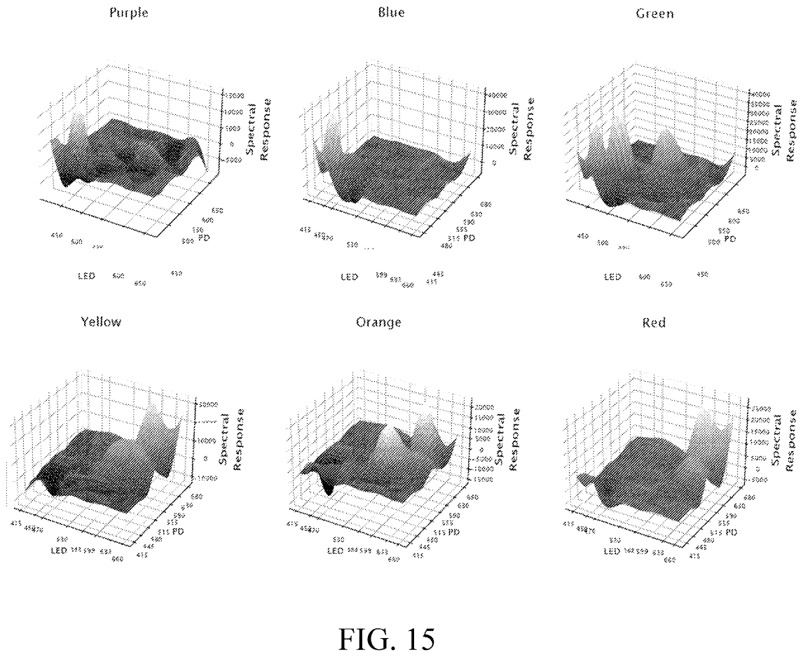

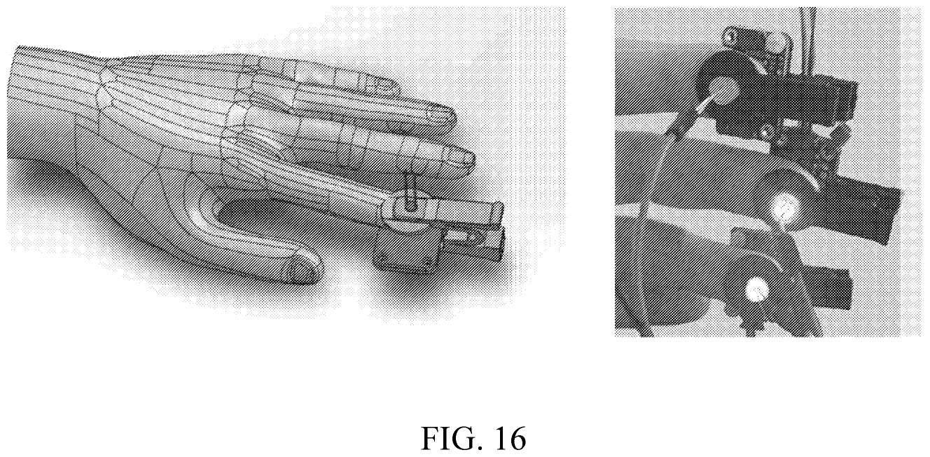

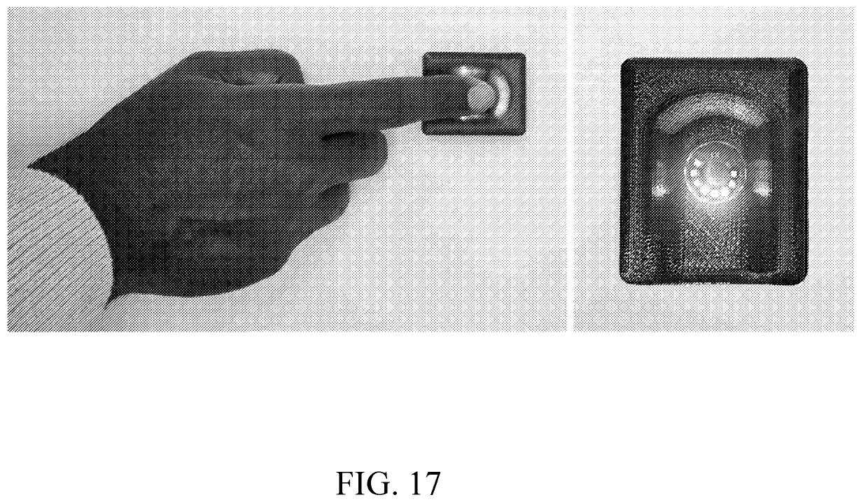

This experimental data collection was done in a controlled lab setting. The AS7341 spectral sensor was placed in a black box with an LED. The ambient light was mitigated as much as possible. When we took readings with the LED off, all channels showed zero counts. The results of this study are shown in FIG. 15. The results from the air medium are shown in FIG. 10, right. We compared the peaks in these figures to the center wavelengths of the filters, demonstrating a mean absolute error of 13 nm with a standard deviation of 8 nm. We further compared this to peak detection before interpolation and showed a mean absolute error of 17 nm with a standard deviation of 11 nm. Thus, we see that interpolation improves our center wavelength detection. In some cases, the filters, such as yellow, covered the entire spectrum after the initial peak, creating an extended peak. We see our algorithm detect this, but as we do not have a center wavelength to compare against, we removed it from our analysis.

### Pilot Study Background and Motivation

Diabetes is a disease that impacts over 400 million people worldwide. It causes a number of complications, including shortened life expectancy, decreased quality of life, blindness, cardiovascular issues, and more. In the United States, over $300 billion yearly is spent to manage and treat diabetes and its numerous complications. Prediabetes is a common precursor to diabetes that is characterized by higher than normal blood sugar levels but lower blood sugar levels than with diabetes. It is possible to treat and even reverse prediabetes through lifestyle changes, but most people with prediabetes are unaware of their condition. To determine if a person has prediabetes, blood glucose tests are run in the clinic. This process is invasive as it requires drawing blood for accurate measurements. Moreover, it is time and resource intensive creating a barrier to prompt testing. By tracking blood glucose via wearable technology, as well as its overall trends, we can potentially monitor the progression of prediabetes outside of the clinic.

Traditional glucose monitoring requires a blood sample and a glucometer. These monitors have been tested, evaluated, and proven effective for diabetes monitoring and management. New devices have come on the market to try to less invasively monitor blood glucose. Continuous Glucose Monitors (CGM) collect interstitial fluid through a needle inserted into the skin to provide continuous glucose monitoring. Breath Ketone Analyzers estimate glucose levels by measuring the ketones in your breath. Research has begun into less invasive optical methods in the visible and near-infrared range that have been done in lab settings and wearable devices. Specifically, studies have shown the fluorescence emission of glucose oxidase peaks at around 520 nm and increases in the presence of glucose in a lab setting. Studies have found that the ratio of the 510 nm wavelength to 475 nm wavelength to be an effective metric for tracking glucose. The found that as glucose concentration increases, the ratio decreases, using Beer-Lambert's law. This work was not in a wearable, but using a light source and smartphone. Additionally, 650 nm wavelength of light being shown on a solution has been shown to increase in transmittance of photons, with glucose and was done using a handheld device. A noninvasive continuous blood glucose monitor can lead to advancements in timely prediabetes identification. The goal of this study is to demonstrate a proof of concept for such a device by leveraging Lumos.

### Spectral Response Identification

In order to identify the optimal wavelengths to monitor glucose, we ran a preliminary study. In this study, we monitored the glucose of a single person over the course of an hour. This study determined that three wavelengths: 470 nm, 515 nm, and 680 nm, showed promising responses for change in glucose. To validate this, we customized our sensor to target 470 nm, 515 nm, and 680 nm. These wavelengths do not align with the original surface mount LEDs used in Lumos. As these components are different, we developed a new custom form factor shown in FIG. 16, left. This form factor was designed to house a single through hole LED while maintaining continuous pressure on the finger. Additionally, this form factor is designed such that it can be worn on three fingers simultaneously. We chose to run each LED on separate fingers to simultaneously collect data from all three sensors without the data loss that switching LEDs could cause. Our prototype is shown in FIG. 16, right.

### Results

We tested our proof of concept sensor in comparison with a commercially available glucometer in a user study. This study used a single participant where we attached the Lumos devices on the participant's left hand as shown in FIG. 16, right. The 680 nm LED was attached to the pointer finger, the 515 nm LED to the middle finger, and the 475 nm LED to the ring finger. Blood glucose readings were drawn from the fingers on the right hand. This setup is shown in FIG. 16, right. Each study was performed over approximately 30 minutes to one hour. We repeated this study five times. Our participant began in a fasted state and we started the studies in the morning. This appears as a low starting glucose readings. When the study began, they drank a sugary drink (soda), and we recorded the effect over time of that on their blood sugar. Approximately every three minutes, we took a new blood glucose reading. We continuously sampled the three Lumos devices at approximately 2 Hz.

In FIG. 2 we show the raw data collected from each LED with its targeted PD. The ground truth blood glucose is shown by the red points. It is measured by a commercially available glucometer. The optical spectroscopy readings from the 480 nm, 515 nm, and 680 nm photodetector channels are shown by the blue lines. In FIG. 2, left and FIG. 2, right, the change in counts positively trends with the change in glucose. In FIG. 2, middle, the change in counts negatively trends with the glucose readings. In this figure, the y-axis of the glucose readings is flipped. We performed further analysis to quantify the correlations between the glucose measurements and the readings from our sensor at our three target wavelengths. To compare these two data points, we interpolated the missing glucose values using a linear interpolation. We found that 470 nm and 515 nm were statistically significant with a p-values less than 0.05 and Pearson correlations of 0.843 and −0.927 respectively. The negative correlation showing that the glucose reading and Lumos's reading are highly correlated but in the opposite directions from each other. 680 nm showed relatively low correlation when compared with our ground truth glucose values with a Pearson correlation of 0.359. The lack of correlation for the 680 nm channel encourages future work in the surrounding wavelengths including expanding into the infrared spectrum. These results show that readings from our sensor may be used to track overarching glucose trends. They prompt additional research to continue to improve the noninvasive monitoring of glucose and thus track the progression of prediabetes.

### GlucoLux

Non-invasive blood glucose monitoring has been a long-sought, elusive goal in diabetes management and healthcare. Traditional glucose monitoring techniques require blood sampling, causing discomfort and inconvenience to users. Optical spectroscopy, which investigates the interaction between electromagnetic radiation and matter, has emerged as a promising technique for non-invasive glucose monitoring. This method enables continuous tracking of glucose concentrations, providing a more comprehensive understanding of an individual's glucose fluctuations. Spectroscopy has already revolutionized medical diagnostics, leading to the development of advanced imaging technologies, such as X-ray, MR spectroscopy, and CT scans. By integrating spectroscopy into a wearable glucose monitoring device, healthcare professionals will be able to access longitudinal data that allows for more informed decision-making and improved patient care.

The integration of wearable technology and optical spectroscopy for non-invasive low-cost glucose monitoring has the potential to significantly impact the lives of both diabetic and non-diabetic individuals. Current solutions for diabetes management require invasive and expensive CGMs (more than $6000 annually) and are reliant on consumable elements that last ten to fourteen days. For diabetic patients, continuous and non-invasive monitoring offers a more affordable and convenient alternative to traditional blood sampling methods, resulting in improved patient compliance, enhanced diabetes management, and easy accessibility. Real-time glucose monitoring empowers diabetic patients to make informed decisions regarding their diet, exercise, and medication, ultimately leading to better glycemic control and a higher quality of life. For non-diabetic individuals, the ability to continuously track glucose concentrations provides valuable insights into their body's response to various activities, meals, and lifestyle choices. This real-time feedback allows them to make data-driven decisions to optimize their health and well-being, potentially preventing the onset of metabolic disorders and promoting overall wellness. The fusion of wearable technology, optical spectroscopy, and advanced data analytics has the potential to transform personal health management, enabling individuals to take a more proactive and informed approach to their health and lifestyle choices.

There is a growing body of research focused on accomplishing noninvasive blood glucose monitoring. Recently, optical sensing, RF signals, electrochemical approaches, and thermal sensing have been leveraged to characterize blood glucose. However, determining blood glucose outside of the lab still remains a challenge. Although these sensing technologies have demonstrated a notable correlation in detecting glucose concentrations in external samples, such as glucose solutions or under controlled laboratory conditions, their effectiveness in accurately measuring blood glucose levels within the body has yet to be substantially proven. In practice, glucose detection on human subjects is an arduous process due to the low concentration of glucose relative to other substances, and the fact that many other factors can affect the measurements. Our approach leverages a larger portion of the electromagnetic spectrum allowing us to discern not only glucose but other compounds in the body that may be creating artifacts or could be used as a reference for standardization across different individuals.

In this disclosure, we address the following, among other things:
How can we noninvasively monitor blood glucose concentrations accurately using optical spectroscopy?
How can we transform noninvasive optical glucose monitoring into a low-cost wearable device that can adapt to dynamic environments and inter-person variability?

In this study, we introduce GlucoLux, a novel optical spectroscopic device designed for the noninvasive measurement of blood glucose levels from the fingertip. Utilizing reflectance spectroscopy, GlucoLux analyzes the interaction between different light wavelengths and glucose in the bloodstream. GlucoLux incorporates components that are combined with our printed circuit board (PCB) as described herein. We evaluated the GlucoLux device on its accuracy when compared with a CGM, its power consumption, and its ability to function as a standalone device. It should be understood that the GlucoLux device is used to illustrate the disclosed technology and that the disclosed technology is not limited to the technology as implemented in GlucoLux.

We developed an algorithm that processes multi-spectrum signals obtained from reflectance spectroscopy and outputs blood glucose values. This algorithm encompasses the data processing pipeline, feature extraction, evaluation, and the machine learning (ML) model used to estimate blood glucose. We evaluated a number of ML models over our dataset and found that we were able to train an Ensemble mode with an R2 of 0.91 and MAE of 3.93 using 5-Fold Cross Validation and an R2 of 0.91 and MAE of 14.66 using a Leave-One-Out Cross Validation. This algorithm constructs a model for glucose estimation that is capable of estimating glucose regardless of variations in physiology and skin tone from person to person. We evaluate our algorithm on its accuracy, mean absolute error, and generalizability. To evaluate our devices we monitored blood glucose with the GlucoLux device and compared it to interstitial fluid glucose readings from a Dexcom G6 (CGM) while calibrating it with fingerstick glucometer readings. Compared with the CGM our device performs favorably within the FDA guidelines. We performed our study over seven participants of varying skin tones to ensure the robustness of our analysis. We found that 98.73% of our estimations are considered to have a high level of clinical accuracy based on a Clarke Error Grid Analysis for 5-fold cross validation and 80% for Leave-One-Out Cross Validation. Additionally, none of our estimations fell into categories that could be considered clinically dangerous. Specifically, we provide herein:

A low-cost wearable optical spectroscopy device for noninvasive glucose monitoring. This device is designed with considerations for human physiology and comfort to make it easily adoptable.

An algorithm to analyze multi-spectrum signals and estimate glucose concentrations in real-time while maximizing accuracy when compared against a commercial continuous glucose monitor.

A user study for glucose concentration monitoring demonstrating the viability of using GlucoLux for continuous monitoring as compared to a commercially available CGM.

### Related Work

Glucose monitoring is critical for 537 million people around the globe suffering with diabetes. Additionally, as of 2021, it was estimated that 762 million people were living with prediabetes. These numbers are expected to continue rising to over 783 million with diabetes and 1 billion with prediabetes by 2045. Of those living with diabetes, it is estimated that 44.7% are currently undiagnosed due to the testing requirements and steep costs associated with CGMs. As such, there is a growing body of research focused on improving glucose sensing technologies to make it less invasive and easily accessible. In this section, we will discuss the current standard of care for glucose monitoring, the progression of the development of noninvasive sensing technologies, and research into noninvasive optical sensing methods.

### Current Standard of Care

Historically, a blood sample is required to detect the concentration of glucose. Traditional blood glucose monitoring is done using glucometers. They are portable devices used to measure blood glucose concentrations. They are commonly used by people with diabetes to monitor their blood glucose concentrations at home. This is referred to as self-monitoring of blood glucose (SMBG). Here is a detailed explanation of how glucometers work: First, a small drop of blood is obtained by pricking the skin with a lancet. The blood sample is typically taken from the fingertip, but other sites, such as the forearm or palm, can also be used. The blood sample is then placed on a disposable test strip, which contains enzymes that react with glucose in the blood. The enzymes produce a small electrical current in proportion to the amount of glucose in the blood. The test strip contains an enzyme called glucose oxidase or glucose dehydrogenase, which catalyzes the oxidation of glucose in the blood. As glucose is oxidized, electrons are transferred, producing a small electrical current that is measured by the glucometer. The amount of electrical current produced is proportional to the amount of glucose in the blood sample. The test strip also contains a chemical called a mediator, which helps to transfer the electrons from the glucose oxidation reaction to the glucometer's measuring system. Different types of test strips may use different enzymes and mediators, but the principle is the same: the enzymes catalyze the reaction of glucose in the blood, producing an electrical current that is measured by the glucometer. The test strip is inserted into the glucometer, which measures the electrical current and calculates the blood glucose concentrations. The result is displayed on the glucometer's screen within a few seconds. After the blood sample is applied to the test strip, the test strip is inserted into the glucometer. The glucometer contains a small electrical circuit that measures the electrical current produced by the test strip. The glucometer converts the electrical current into a numerical value representing the blood glucose concentrations and displays the result on its screen within a few seconds. Some glucometers may also store the results in memory, allowing the user to track their blood glucose concentrations over time. Further, smartphones can now be leveraged to read a glucose strip.

On the other hand, a continuous glucose monitor (CGM) is a device that measures glucose concentrations in the interstitial fluid (ISF), which is the fluid that surrounds the body's cells. CGMs are used to monitor glucose concentrations in people with diabetes and are typically worn on the body, such as on the abdomen or upper arm. A small sensor is inserted into the subcutaneous tissue (the tissue just beneath the skin) to measure the glucose concentrations in the interstitial fluid. To measure glucose concentrations in the interstitial fluid, a CGM system consists of three main components: a glucose sensor, a transmitter, and a receiver. The glucose sensor is a tiny electrode that is inserted just under the skin using a small needle or insertion device. The sensor measures glucose concentrations in the interstitial fluid every few minutes using a process called enzymatic electrochemistry. Specifically, an enzyme on the sensor reacts with glucose in the interstitial fluid, producing a small electrical current that is proportional to the amount of glucose present. The enzyme that is typically used in a CGM sensor to react with glucose is glucose oxidase. This enzyme catalyzes the oxidation of glucose to gluconic acid, producing hydrogen peroxide as a byproduct. The hydrogen peroxide is then detected by the sensor and converted into an electrical signal that can be measured and transmitted wirelessly to the receiver. To ensure accurate glucose readings, CGMs require calibration with a blood glucose meter. The user must perform a fingerstick and enter the blood glucose reading into the CGM. The CGM then adjusts its readings based on the calibration value. Compared to a blood glucometer, a CGM provides continuous glucose monitoring and can detect trends in glucose concentrations over time, whereas a blood glucometer only provides a snapshot of the glucose concentrations at the time of measurement. CGMs also typically require fewer fingersticks than blood glucometers, as the sensor is inserted under the skin and measures glucose continuously. Additionally, smartphones can leverage lifestyle information to estimate glucose concentrations, extending the utility of CGMs. However, CGMs may have some limitations, such as a delay between changes in blood glucose concentrations and changes in interstitial fluid glucose concentrations, and may not always provide accurate readings in certain situations, such as during rapid changes in glucose concentrations or during exercise.

Glucose is found throughout the body including in blood, ISF, sweat, lacrimal fluid, saliva, and urine. In each fluid, glucose exists in varied concentrations. Further, glucose presents in interstitial fluid, urine, sweat, saliva, and lacrimal fluid with a time delay and less pronounced high and low concentrations. In this section, we discuss alternative bodily fluids, sweat, lacrimal fluid, saliva, and urine, in which glucose is present. We describe the relationship between blood glucose concentrations and the concentration of glucose in each fluid. Further, we detail the approach to noninvasively monitor the glucose concentrations in each fluid.

Saliva is a clear fluid secreted by the salivary glands of the mouth. It is mostly water, but it also contains a number of elements important for digestion, immunity, and barrier protection. Additionally, saliva contains a variety of biomarkers, including glucose. Glucose appears in saliva when there is a surplus in the body, and even then, it is only present in very low concentrations when compared to concentrations in the blood. These concentrations can potentially miss life-threatening low blood glucose readings. Even then, there is a 30-40 minute delay in observed changes in salivary concentration when compared to blood concentrations. A saliva based glucose sensor generally takes the form of a mouthguard to noninvasively collect samples. While in a controlled environment, it shows promising results, in real-world use, there is interference from food, biofilm accumulation on the sensor, and a variety of other biomarkers. Overall, salivary glucose concentrations may be useful for long-term chronic management (identifying high glucose concentrations), but they lack both monitoring of a dynamic concentration as well as detection of critical low values.

Lacrimal fluid (tears) is a clear fluid secreted by the lacrimal glands around the eye. It is mostly comprised of water but functions to hydrate and nourish the eye, provide immune protection, and prevent fungal growth. It contains a variety of biomarkers, including glucose, and tends to have low concentrations of interference from other sources. The external membrane of the eye relies on nourishment from the lacrimal fluid, including glucose. As such, glucose needs to be present in lacrimal fluid. Even as blood glucose concentrations decrease, the amount of glucose present in the lacrimal fluid does not decrease proportionally, obfuscating life-threatening low blood glucose concentrations. As glucose concentrations in the blood increase, the amount of glucose present in the lacrimal fluid increases as well. This fluid experiences greater than a ten-minute delay when compared to blood glucose. Lacrimal fluid-based glucose sensors are typically incorporated into contact lenses. These sensors show promising results in controlled environments and lower accuracies in real-world trials. Moreover, contact-based sensors experience limitations with respect to circuit design, power supply, and the production of heat that can cause irritation to the eye. Altogether, lacrimal fluid glucose concentrations would be favorable for identifying persistently high glucose concentrations but would be incomplete as a comprehensive glucose monitor.

Urine is a clear to yellow fluid that is excreted from the body as the primary mechanism to manage hydration and electrolyte balance. It has a long history of being used to monitor biomarkers, including glucose. Glucose can be found in urine when blood glucose concentrations exceed the capacity of the kidneys to recapture the glucose. But due to the 20-minute to 2-hour time delay along with the intermittent nature of the collection of this fluid, it does not make an ideal fluid for continuous monitoring. Overall this fluid is suited for diagnosis-based testing for diabetes in clinic, where it has extensively been used in the past.

Sweat is a clear fluid secreted from sweat glands in the skin conveniently located all over the body. Its primary function is thermoregulation. Sweat contains a variety of biomarkers, including glucose. Glucose is continuously present in sweat, and since sweat glands are highly vascularized, it is possible to interpolate blood glucose. The concentration of these biomarkers is dependent on a number of factors, including rate of sweating and sweat glucose exhibits a 10-minute delay from blood glucose. One of the complications of long-term diabetes is that sweating becomes dysregulated. Due to this, the glucose concentrations in their sweat can become diluted due to an overproduction of sweat or overly concentrated due to an underproduction of sweat. Further complicating this, non-sweat-related sources of glucose on the skin can confound glucose measurements. Despite these challenges, many noninvasive sweat glucose sensing devices have been developed in the form of a watch, patch and even nose-bridge pads on eyeglasses. While these sensors achieve acceptable results in controlled settings, they fall victim to the many confounding factors that they encounter in real-world settings. Overall, sweat-based glucose sensing is a promising modality as it has the ability to detect not only high blood glucose concentrations but also low blood glucose concentrations, but unfortunately, it is currently severely limited by difficulties continuously collecting a sample, many interfering impurities in collected samples, and differing physiologies.

Transdermal glucose sensing is an elusive challenge. It estimates blood glucose concentrations without the need for an invasive sample. While it does not require the collection of biological fluids or invasive sensors, it presents a much more challenging target, as direct measurement is not possible. A number of different solutions have been attempted, including thermal sensing, electrochemical sensing, electromagnetic sensing, polarimetry, and optical coherence tomography. However, these techniques have many confounding factors, including but not limited to environmental conditions and tissue perfusion leading to insufficient accuracy and reliability. In this section, we will focus on optical sensing in the visual and infrared spectra.

Near-infrared spectroscopy (NIR 680-2500 nm) and mid-infrared spectroscopy (MIR 2500-25000 nm) measure the absorption and emission of these wavelengths given a medium. In lab settings, these sensors have seen promising results given glucose diluted in an aqueous solution. But in real-world on-body applications, these sensors fall victim to the complexities of human tissues and the superposition of spectral information from these complex tissues. Near-infrared sensors are also present in commercial smartwatches, making them an ideal candidate for sensing. We leverage NIR wavelengths in our sensor to complement our visual spectral sensors.

In addition to the advances made in research, there are several commercial noninvasive glucose monitors coming to market. Devices from GWave, UBand, and glucoWISE leverage RF sensing, but their accuracies, when compared to SMBG and CGM, are still limited. SugarBEAT utilizes reverse iontophoresis to noninvasively sense ISF glucose through the skin but has fallen short on accuracy and, thus, adoption when compared to traditional invasive CGMs.

Visible light spectroscopy has been explored with regard to glucose concentrations. In lab settings, glucose has been found to exhibit absorption simultaneously in multiple areas of the visible spectrum. While many of the systems that deploy multiple wavelength approaches find promising results, they still fall victim to confounding factors. Each system has its own unique combination of light emitting diodes (LEDs) and photodetectors (PDs). Frequently, these systems have PDs that appear near 530 nm, 650 nm, and 850 nm. Some include a PD near 470 nm or even higher NIR wavelengths. Our approach leverages the promising results of the multiple wavelength approaches across both visible and infrared light. We utilize a range of LED and PD combinations across the entire targeted spectrum to not only target glucose with the wavelengths mentioned above but also to identify portions of the spectrum that can be used to eliminate or mitigate confounding factors. Overall, optical spectroscopy offers a noninvasive method for monitoring blood glucose, making it a valuable tool for research, clinical, and commercial applications.

### GlucoLux Prototype

When designing the GlucoLux sensor, we chose an on-body location that would be most likely to yield positive results when using reflectance spectroscopy. Because of this, we considered blood flow to the area and how likely users would be able to comply with wearing our device. The fingertips, toes, earlobes, and forehead—have a relatively high perfusion index because of their rich vascularization. While all of these locations demonstrate high perfusion, the fingertip provides ease of access, familiarity, standardization, and less skin tone variation. Fingertips are easily accessible and usually less affected by peripheral vasoconstriction, a condition where the body decreases blood flow to the extremities in response to cold temperatures or stress than other parts of the body. As they are easily accessible, they are well studied and have become adopted by spectroscopy-based devices like a fingertip-based pulse oximeter. Further, melanin concentration is relatively lower on the fingertips when compared to the earlobe, wrist, and forehead. Traditionally, transmittance spectroscopy is used for the fingertip but for future transition towards a wrist-based location we choose reflectance spectroscopy.

Non-invasive glucose monitoring using optical spectroscopy has been a topic of significant research interest. The basic principle involves using light to probe the tissues and analyzing the reflected, transmitted, or absorbed light to infer glucose levels. Different techniques include near-infrared (NIR) spectroscopy, Raman spectroscopy, and photonic crystal technology among others. Glucose is only one of many substances in the body that interacts with light, and it is present at relatively low concentrations (700-1400 ppm). This makes it difficult to distinguish the signal due to glucose from the signals due to other substances. Moreover, various factors such as temperature, hydration, and tissue properties can also affect the measurements. In lab settings, glucose has been shown to have absorption peaks at four wavelengths, in particular, 485 nm, 645 nm, 860 nm, and 940 nm. However, in practice, this is tedious due to the low concentration of glucose relative to other substances, and other factors such as metabolism, motion artifacts, etc. In our studies, we found these wavelengths to be correlated with glucose, but it was not consistent across all individuals. Thus, we chose a more comprehensive set of LEDs and PDs to capture more of the spectrum and hence more information on the other factors in the body.

### Prototype Design

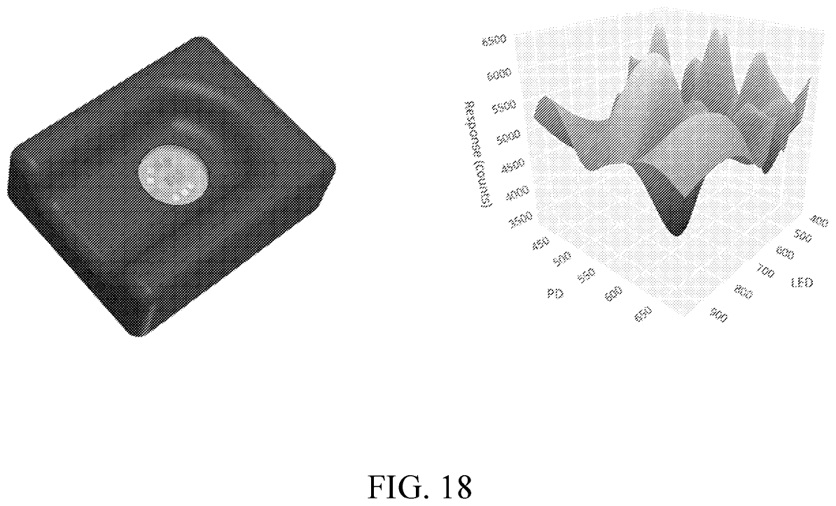

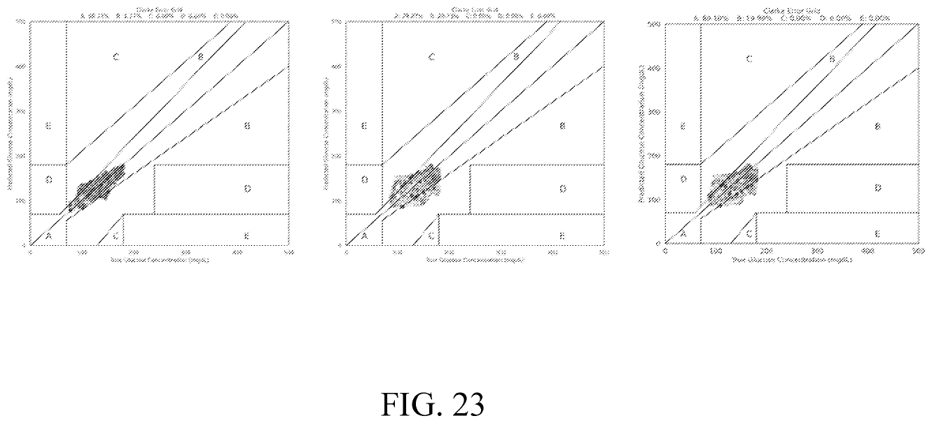

The GlucoLux device represents a cutting-edge advancement in open-source wearable glucose monitoring technology. It is a multi-spectra optical finger sensor, meticulously designed for both form factor and usability as seen in FIGS. 23, left, and 19. Central to its functionality is the integration of a microcontroller with an LED driver, battery, and optical sensor, all on a single PCB.
Microcontroller & LED Driver: At the center of the device is an nRF52840 chip, featuring an ARM® Cortex®-M4 microprocessor. This chip communicates with the LED driver and PD via SPI and I2C protocols respectively. Its compact, single-sided surface-mountable design is ideal for wearable applications. This setup enables low-power BLE (Bluetooth Low Energy) and TinyML implementation, facilitating efficient data collection and on-chip processing. The microcontroller's battery management system is crucial for making the device both remote and reusable. The TLC59711 12-channel LED driver modulates the current supplied to each LED, enabling precise control over their lumen intensity. This feature is crucial for normalizing spectral signals across users with varying skin tones and conditions.
Battery: The device is powered by a 400 mAh Li—Po battery, which distinguishes it from single-use CGMs currently on the market. This battery allows for up to four hours of continuous usage, ensuring longevity and sustainability in monitoring applications. The battery life of the device can be extended by either providing a larger battery or duty cycling the sensor based on the needs of the application. In its current implementation, a new reading is recorded approximately every three seconds. For a real-world glucose monitoring device, sampling every five minutes, similar to a CGM, would be sufficient.
Optical Sensor: The optical sensor array consists of 9 LEDs, each with a lumen range of 104-114 and wavelengths varying from 415 nm to 910 nm. The AS7341 PD is an 11-channel sensor capable of detecting wavelengths between 350 nm and 1000 nm at a sampling rate of 0.3 Hz. This arrangement not only captures the reflected value for each wavelength but also creates a comprehensive LED-PD pair as seen in FIG. 18, right for multi-spectral data analysis.

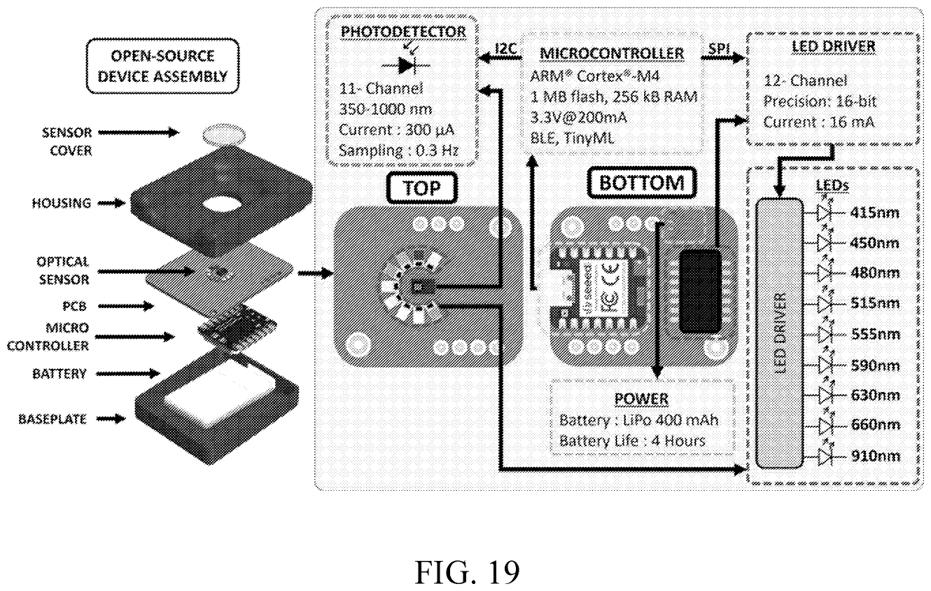

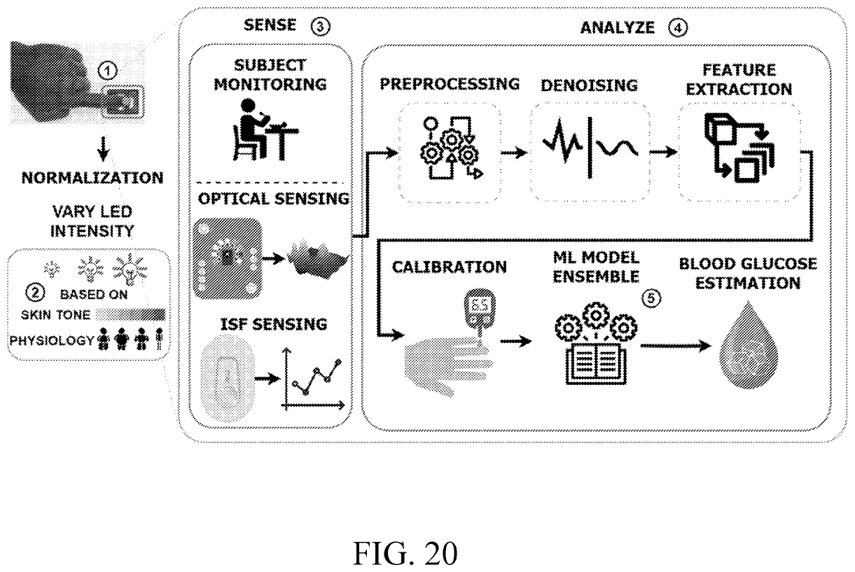

A detailed view of the device is presented in FIG. 19, showcasing the intricate layout and compact design of the GlucoLux device, with dimensions of 44×35×15 mm. This integration of components on a single PCB allows for the simultaneous sensing of multi-spectrum signals, collection of raw signal values for each wavelength, and processing of spectrum data, all within the same compact device.

### GlucoLux Algorithm

Data collected from the device is retained in the form of raw analog-to-digital converter (ADC) counts, each corresponding to an individual PD channel reading for a particular LED activated in time. We consider a single temporal data point in the device's operation to be defined by the count measurements for each LED-PD pair.

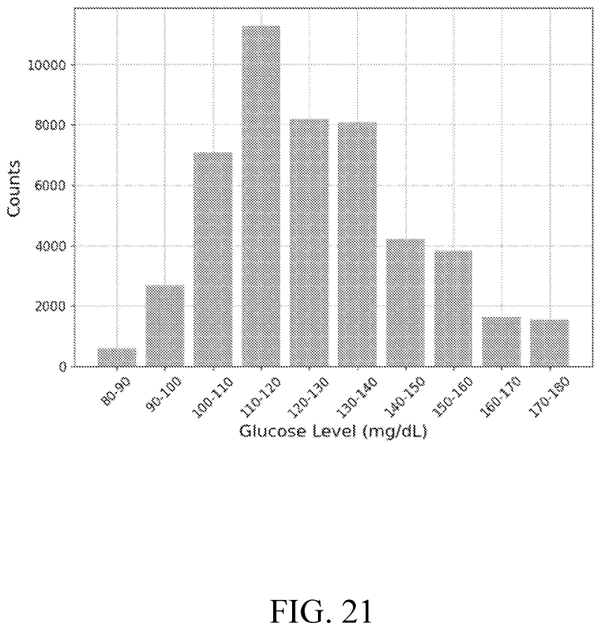

The device consists of 11-channel PD and 9-LEDs, resulting in a combination of 99 LED-PD values. Consequently, it becomes necessary to process the data such that each LED-PD measurement is ‘flattened’. In this context, flattening ensures that a single temporal data point encompasses the PD counts for every LED measurement within a complete cycle, where a cycle is defined as the activation of every LED. CGM data by the Dexcom device is retrieved from each subject's account portal. The 7 subjected CGM glucose value distribution can be seen in FIG. 21. The glucose readings are predominantly distributed over 110 mg/dL-120 mg/dL as the data was collected during each meal resulting in blood glucose spikes. A range of 78 mg/dL-180 mg/dL is chosen for this study based on FDA guidelines for acceptable CGM readings.

While the developed sensor is relatively robust to motion artifact and noise, there was observed interference particularly from significant motion, user/device error, and sensor instability during initial testing. As such, data pre-processing pipelines were required to filter out motion artifacts and detect anomalies within the data. The methodology to deal with this interference was two-fold. Firstly, motion artifacts or spikes within the data were smoothed using a 20-point moving average filter. Secondly, following post-processing, anomalies were detected and flagged by analyzing the rolling mean and the rolling standard deviation in a 20-point sliding window; if the current sensor value is outside the rolling mean by more than two standard deviations, it is flagged as an anomaly. This method proved to be far more robust relative to other measures such as interquartile range, given behavioral changes of the sensor observed experimentally from subject to subject. Anomalous data sets were manually inspected and cleaned as appropriate.

### Interpolation

A significant difference exists between the sampling frequency of the CGM (every 5 minutes) and the GlucoLux sensor (every 3 seconds). Given the sparsity of the CGM data, it was required to interpolate the CGM data to match the GlucoLux sampling rate or downsample the GlucoLux data to match the sparse CGM data. It was previously determined that interstitial glucose concentrations are more stable than blood glucose concentrations. This stability suggests that, despite the low sampling rate of the CGM, it is unlikely that large transient spikes would exist between measurements. Since GlucoLux data is measuring blood glucose, the underlying dynamics are likely far less stable, and it was more reasonable to interpolate the CGM data in order to capture trends in the GlucoLux data that may be lost with downsampling. We utilized cubic spline interpolation as a method to achieve this. Cubic spline interpolation is an effective technique for estimating missing data points within a known range. The computation consists of fitting cubic polynomials to the existing data, which is then used to estimate the missing values. This interpolation method is preferred over other data-filling methods, such as linear interpolation or mean imputation, because it preserves the overall trend and fluctuations in the data and minimizes the introduction of artificial variability or bias, thus providing a more accurate and realistic representation of the underlying biological processes.

Normal use of a CGM requires regular calibration via a finger stick blood sample processed by a glucometer. While calibration assists the CGM with providing more accurate glucose readings, it also can initiate a rapid change in the CGM's readings. For example, we saw calibrations off by upwards of 40 mg/dL, when compared to a glucometer reading. This led to a rapid change in the CGM readings when there was no rationale for the underlying physiology to have changed so dramatically. Due to this incongruity in the CGM and the glucometer reading, these situations can confound any future model by mapping two potentially vastly different glucose measurements to very similar GlucoLux readings. To flag and remove these scenarios, we implemented a simple slope detection on the glucose readings. If a nearly vertical slope was detected, the preceding data points were omitted from our dataset. We chose the preceding data points for omission as they were more likely representative of a period of time where the CGM had diverged from the ground truth. Rather than excluding all data prior to the calibration, this allows retaining a larger dataset by preserving the data in which the CGM and the glucometer were congruent.

Data synchronization is crucial in building a blood glucose estimator that is trained using a CGM that measures ISF glucose levels. CGMs measure ISF glucose concentrations which can be used to estimate blood glucose concentrations by understanding physiologic delays present between them. Traditionally, the physiologic time delay (τ) between blood glucose (BG) and interstitial fluid glucose (IG) at time (t), can be modeled by a two-compartment diffusion model:

$$\begin{matrix}τ\_IG ( t ) \\ \partial t\end{matrix} = - IG ( t ) + BG ( t ) \qquad ( 1 )$$

While this model does reflect the relationship under ideal circumstances, it is limited in a number of common physiologic scenarios, for example, physical activity, hypoglycemic events, and meal-related hyperglycemia. Physiologically, when glucose exists in high concentrations in the blood and low concentrations in the tissue/ISF, glucose will move down its concentration gradient from the blood into the ISF. This process is not instantaneous and will vary from person to person but also based on metabolic demands. The glucose concentration in the ISF depends more on the demands of the surrounding tissues than the concentration in the blood itself. For example, the blood supplies glucose to the ISF, but if the surrounding tissues have high demands the ISF concentration may not accurately approximate the blood glucose concentrations.

Contrary to the CGM, the GlucoLux device is designed to target the blood glucose in the capillary-rich beds of the fingertip. While there may be signal contamination from the glucose in the other tissues, the expected main source of glucose is blood. Thus, it is not a direct translation to align the CGM time (ISF) to the GlucoLux time (blood). Further, the GlucoLux signal is not one-dimensional, but a multi-dimensional signal made up of many features. To synchronize the CGM and GlucoLux data, we employ Multi-Dimensional Dynamic Time Warping (MD-DTW). MD-DTW leverages all dimensions of the signal to find the best time synchronization, as it assumes that the synchronization information will be spread over a number of dimensions. As such it has become a popular choice for synchronization of multi-modal sensor data. While MD-DTW itself has high computational complexity, it is only used to align the ground truth for analysis and does not need to run on our GlucoLux device. On average, we calculated a time delay of 7.48 minutes with a standard deviation of 2.78. We compared this by using the traditional diffusion model, which yielded a mean value of 15.98 minutes and a standard deviation of 14.1 minutes. With the MD-DTW method, we saw a time delay within the reported range of 4-25 minutes. The traditional model did not perform as well with time delays well outside the reported range.

TABLE 2
Derived Features from GlucoLux Measurements

| Feature | Description |
|---|---|
| Raw ADC Counts | Raw spectral data from each LED-PD |
| | combination |
| Successive | Difference between two successive |
| differences | LED-PD spectral values in time |
| RMS successive | Root mean square of successive |
| differences | differences |
| Rolling window | Mean and median calculated over |
| statistics | varying temporal windows LED-PD |
| Ratios | Ratios of each LED-PD measurement |
| | with another overtime |

A multitude of features were engineered from the resultant flattened data structure. Each feature was inspired by methodologies drawn from traditional statistical measures, heuristics, and common signal-processing techniques. These methodologies are commonly utilized in optical sensing applications, such as photoplethysmography.

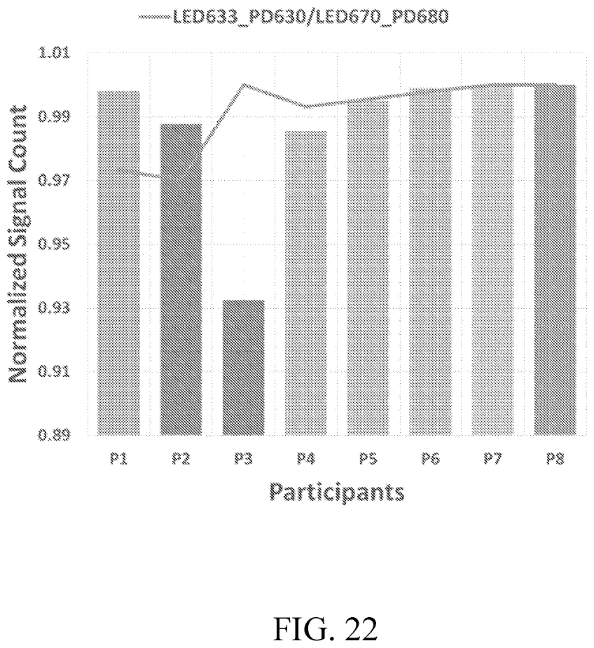

(PPG). A non-exhaustive list of the tested features, along with their descriptions, can be found in Table 2. These features can experience large interpersonal variability and environmental interference that may cause instability in the ADC counts from test to test and person to person. To remedy this, we devised relative measures that we found to be more robust features. We illustrate this in FIG. 22 where we plot the count value for a specific LED-PD compared to one of its relative measures. In this figure, we focus on the LED emitting light at 633 nm wavelength being observed by the PD at 630 nm wavelength. For each participant, we display their skin tone as the color of the bars. In the case of this LED-PD, the melanin concentration in the skin affects the spectral values where different skin tones absorb and reflect light with varying counts. To normalize this we divide each LED-PD pair with another LED-PD pair resulting in the same ratio with respect to counts. This is shown by the green line for the ratio LED633_PD630/LED670_PD680 which shows 85% less standard deviation in comparison to the single LED_PD pair (LED633_PD630). Similar results were seen across the many other relative measures that can be calculated from the signal. Ultimately, relative measures were utilized as features for the ML model given their stability from test to test, reduction in inter-person variability, strong and consistent correlations across subjects, and their analogous use in measures such as pulse oximetry in PPG.

We initially extracted a comprehensive set of 4095 features derived from the 99 multi-spectral signals captured by our device. However, to optimize our model's performance, we recognized the necessity of refining this feature set. We reduced the number of features to 245 through the application of the Gini impurity criterion, focusing on features with a threshold magnitude greater than zero. This criterion was consistently applied across three advanced machine learning algorithms: LightGBM, CatBoost, and XGBoost. The final selection of 245 features represented the intersection of significant features identified by all three models. This strategic reduction in features played a pivotal role in enhancing our model's accuracy. It effectively minimized the error rate by preventing overfitting, eliminating misleading features, and significantly improving the interpretability of the model.

### ML Models

In order to predict glucose levels, we use a supervised learning approach by building predictive models, where we employ multivariate features (I) derived from GlucoLux measurements to learn a predictive function (H).

$$H ( I = [ {{f}_{1}}^{\to -} , {f}_{2} , … , {f}_{n} ] ) - \to \{ gl ∈ R \vert gl > 0 \} \qquad ( 2 )$$

The Eqn. 2 represents the predictive function (H) accepting an input→-/and gives the prediction output g\hat{l}. Since glucose level (gl) is a continuous value. Therefore, predicting the continuous output value gl is a regression problem.

In order to build a robust and generalizable regressor, we train different numbers of regressors like Linear regressor, Ridge regressor, RandomForest, XGBoost (Extreme Gradient Boosting), Light Gradient Boosted Machine (LightGBM), Category and Boosting (CatBoost), Multi-layer Perceptron (MLP), and Convolutional Neural Network (CNN).

RandomForest is a tree-based algorithm that fits a number of decision tree regressors on several subsamples of the dataset to minimize overfitting and utilizes averaging to improve the prediction.

XGBoost uses multiple parallel gradient-boosted decision trees by applying newton boosting, extra randomization parameter, and penalization of trees cleverly to improve efficiency and performance.

LightGBM employs a tree-based algorithm such as gradient-based one-side sampling and exclusive feature bundling for fast, and accurate inference.

CatBoost uses symmetric trees to speed up inference and employs ordered boosting to avoid over-fitting to offer a better quality model.

MLP model consist of an input layer, at least one hidden layer, and an output layer, which learns linear and non-linear relationships between the input and output.

To make our model more robust and generalizable, we embrace ensemble learning, where we utilize top-n performing regressors and obtain the prediction of all regressors and then take average of it. Such an ensemble of different regressors, despite its simplicity offers a best performing model. The simplistic nature of the model allows for easy integration for on-chip glucose estimation using TinyML on the microcontroller. The basic notion is to build a few good predictors and combine them into an even better predictor and the same thing we have employed in our experiment. Finally, to ensure generalizability we perform a 5-fold cross validation and Leave-One-Out cross validation (LOOCV). With respect to the LOOCV we additionally focus on personalization of the model through an initial calibration step to improve accuracy.

### Evaluation

We evaluate the accuracy of GlucoLux using data collected in an N=7 user study. We recruited seven participants (four male, three female) with a range of skin tones as seen in FIG. 22 to improve and test the generalizability of our model. Researchers helped participants apply a Dexcom G6 Continuous Glucose Monitor on their nondominant arm and instructed them on the appropriate use of the monitor, as well as the steps for calibration via glucometer. The participants wore the CGM continuously for a period of 2-3 days. As the GlucoLux device is set up for fingertip monitoring, we asked participants to wear the device during their regular mealtimes. We do this to observe a physiologic fluctuation in their blood glucose levels in a real-life environment. Each participant was asked to collect 5 hours of data using the GlucoLux device, accounting for approximately 3-4 meals per person. In total, we collected approximately 42 hours of data, and approximately 6 hours were removed due to a malfunction of the case the device was housed in becoming malpositioned and obscuring our 415 nm LED. To ensure that the CGM glucose readings are uniformly distributed we focus on glucose reading within 78-180 mg/dL.

To evaluate the performance of the models, we have used metrics: R2-Score (Coefficient of Determination), Mean Absolute Error (MAE) and Mean Absolute Percentage Error (MAPE). The model is evaluated using a 5-Fold Cross Validation (5-Fold CV) and a Leave-One-Out Cross validation (LOOCV). The 5-fold CV allows for reduced variance in evaluation while the LOOCV tests the model's ability to generalize to entirely new subjects, which is crucial for medical applications where the model needs to perform well on unseen patients. The reason to choose R2-score is, it measures the strength of the relationship between the regressor and the dependent variable and also offers an indication of goodness of fit. Also, it conveys how well unseen samples are likely to be predicted by the regressor via the proportion of explained variance. The R2-score is defined as

$$R 2 = 1 - \frac{{{{I}^{′}}_{t = 1}^{N} ( {gl}_{i} - g {\hat{l}}_{i} )}^{2}}{{{{I}^{′}}_{t = 1}^{N} ( {gl}_{i} - g {\overline{l}}_{i} )}^{2}} \qquad ( 3 )$$

TABLE 3
Experimental results for tree-based regressors using 5-fold cross-validation
on training and validation set employing only top 245 significant features.

| | Training Set | | | Validation Set | | |
|---|---|---|---|---|---|---|
| Regressor | R2 | MAE | MAPE | R2 | MAE | MAPE |
| LightGBM | 0.92 (±0.0006) | 3.85 (±0.0249) | 0.03 (±0.0002) | 0.91 (±0.0032) | 4.11 (±0.0549) | 0.03 (±0.0005) |
| CatBoost | 0.92 (±0.0008) | 4.09 (±0.0197) | 0.03 (±0.0002) | 0.89 (±0.0034) | 4.45 (±0.0665) | 0.04 (±0.0006) |
| XGBoost | 0.92 (±0.0004) | 3.77 (±0.0128) | 0.03 (±0.0001) | 0.91 (±0.0027) | 4.14 (±0.0570) | 0.03 (±0.0006) |
| RandomForest | 0.93 (±0.0001) | 3.43 (±0.0261) | 0.03 (±0.0002) | 0.90 (±0.0037) | 4.10 (±0.0624) | 0.03 (±0.0006) |
| MLP | 0.87 (±0.0091) | 5.44 (±0.2317) | 0.04 (±0.0020) | 0.87 (±0.0101) | 5.49 (±0.2045) | 0.04 (±0.0016) |
| Ensemble | 0.93 (±0.0006) | 3.53 (±0.0124) | 0.03 (±0.0001) | 0.91 (±0.0031) | 3.93 (±0.0608) | 0.03 (±0.0006) |

While MAPE is a mean absolute percentage deviation metric, that is sensitive to relative errors and not impacted by a global scaling of the dependent variable. The MAPE is

$$MAPE = \frac{1}{{N}_{i = 1}} \frac{{\sum }^{N} \vert {gl}_{i} - g {\hat{l}}_{i} \vert}{{gl}_{i}} \qquad ( 4 )$$

Finally, MAE is used because it provides a clear and straightforward measure of the average error magnitude in the device's glucose readings. This is particularly relevant in the context of the U.S. FDA guidelines

$$MAE = \frac{1}{{N}_{i = 1}} {\sum }^{N} \vert {gl}_{i} - g {\hat{l}}_{i} \vert \qquad ( 5 )$$

In our research, we focus on assessing the accuracy of predicted glucose level values (g\hat{l}) against actual glucose levels (gl), with consideration given to the mean glucose level (gl̅) across a dataset of ‘N’ total samples. We employed a K-folds (K=5) cross-validation method for a comprehensive analysis. This involved dividing the dataset into five equal-sized subsets, or ‘folds’. Each fold was used once as a validation set, while the remaining folds collectively formed the training set. We trained the model on these subsets and then evaluated it on the validation set. This process was repeated until each fold had served as a validation set, ensuring that every data point was utilized in both the training and validation phases. The model's performance was assessed by averaging the scores obtained from each cycle, with detailed results presented in Table 3 for various regressors. This cross-validation approach minimizes biases associated with data partitioning, ensuring a thorough and unbiased evaluation of the model's performance across the entire dataset.

To compare our model's prediction to the CGM's output in a clinically meaningful manner, we utilized Clarke Error Grid Analysis (EGA). EGA is a vital tool in the field of glucose monitoring and diabetes management that provides a quantitative method of evaluating the clinical accuracy of glucose monitoring systems. The evaluation involves comparing each measurement against a reference measurement and considering the clinical consequences of prediction inaccuracies. The analysis divides the comparison space into five zones: A, B, C, D, and E. The significance of each zone is as follows:
Zone A: Clinically accurate measurements that fall within 20% of the reference sensor.
Zone B: Measurements outside of the 20% threshold, but would not lead to inappropriate intervention.
Zone C: Measurements that would lead to inappropriate intervention (overcorrection or undercorrection of glucose levels).
Zone D: Measurements that would lead to inappropriate actions with the potential for harm. Zone E: Measurements that would lead to erroneous actions and significant health risks.

To create a reliable and effective glucose sensor, the overarching goal in this evaluation is to ensure that the majority of readings fall within Zone A and B of the EGA, while avoiding Zones D and E. FIG. 23 presents our EGA analyses on seven different cohorts; the first six on six unseen sets (a-f), and the last on testing, training, and validation datasets for all subjects (g). The publicly available package ‘glucose-stats’ was used to generate these grids.

Based on this analysis, we found that our validation all subjects using 5-fold CV resulted in 98.73% of our estimated glucose data points fall within group A, indicating a high level of clinical accuracy. Additionally, 1.28% fall within group B, with no values in group C, D, or E, suggesting that the sensor remains relatively stable and precise within the normal range of glucose measurements. For our LOOCV with and without calibration, 100% of all data points fell within groups A and B, with 80% falling in group A. This level of performance underscores the reliability and effectiveness of the sensor across a range of subjects, skin-tones, and environments during a single measurement. Generally, while a single erroneous reading (such as one in Zone C) can be potentially concerning, the output readings of the sensor can be smoothed before reporting to avoid inaccurate spikes and a potentially anxiety-inducing glucose reading. The high sampling rate of our device allows for sufficient trend capturing such that smoothing would not impact any true transients observed for measurements in a real-world clinical setting. Given this data, it is reasonable to assume that, given the high sampling rate and clinical accuracy of the device, the sensor can provide medically meaningful and accurate results not only during a single point of time, but generally over the course of its use over time. The device's performance in a LOOCV with calibration falls within the FDA guidelines for CGM measuring within 70 mg/dL to 180 mg/dL. Table 4 shows that LOOCV with calibration has a MAE less than 15 mg/dL which is the commercial requirement by the FDA.

TABLE 4
Leave-One-Out Cross Validation Result
with and without Calibration

| | MAE | MAE | MAPE | MAPE |
|---|---|---|---|---|
| | Mean | STD | Mean | STD |
| LOO CV(Calibration) | 14.66 | 2.47 | 0.109 | 0.015 |
| LOO CV(w/o Calibration) | 15.24 | 3.13 | 0.120 | 0.025 |

Our prototype evaluation encompasses a critical assessment of the system's size, computational ability, and battery life. This assessment evaluates the devices ability to be deployed in the real world. Sufficient battery life, computing power, and a small light form factor are critical to device adoption and use. The GlucoLux device measures 44×35×15 mm and capable of sensing and estimate glucose levels without the need of external power supply or computation. The GlucoLux device cycles through all nine LEDs in a span of three seconds, ensuring a rapid and comprehensive data collection process. Additionally, feature computation is highly efficient, with the device able to calculate the 245 features within a mere 4 ms. The GlucoLux device, powered by the nRF52840 chipset, comes equipped with 1 MB Flash memory, of which our ensemble model utilizes a modest 16 KB (before feature optimization). The 256 KB SRAM comfortably accommodates the input parameters, which require 2 KB for the 245 features. Our assessments confirm the feasibility of running the ensemble model using TinyML on the nRF52840, demonstrating the device's robust computational ability. The GlucoLux device consumes between 100-150 mW to operate the reflectance device with on-chip TinyML, without the need for data transmission to servers. Utilizing a 400 mAh battery rated at 3.7V, we estimate approximately 4 hours of operational battery life. This considerable battery longevity enhances the GlucoLux device's potential for continuous, real-time monitoring applications.

All ranges disclosed herein are inclusive of the recited endpoint and independently of the endpoints. The endpoints of the ranges and any values disclosed herein are not limited to the precise range or value; they are sufficiently imprecise to include values approximating these ranges and/or values.

As used herein, approximating language can be applied to modify any quantitative representation that can vary without resulting in a change in the basic function to which it is related. Accordingly, a value modified by a term or terms, such as “about” and “substantially,” may not be limited to the precise value specified, in some cases. In at least some instances, the approximating language can correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” can refer to plus or minus 10% of the indicated number. For example, “about 10%” can indicate a range of 9% to 11%, and “about 1” can mean from 0.9-1.1. Other meanings of “about” can be apparent from the context, such as rounding off, so, for example “about 1” can also mean from 0.5 to 1.4. Further, the term “comprising” should be understood as having its open-ended meaning of “including,” but the term also includes the closed meaning of the term “consisting.” For example, a composition that comprises components A and B can be a composition that includes A, B, and other components, but can also be a composition made of A and B only. Any documents cited herein are incorporated by reference in their entireties for any and all purposes.

### Aspects

The following Aspects are illustrative only and do not limit the scope of the present disclosure or the appended claims. Any part or parts of any one or more Aspects can be combined with any part or parts of any one or more other Aspects.
Aspect 1. A method for determining levels of a biomarker of a user by a mobile device, comprising: generating a first light comprising a first wavelength; collecting emission signals resulting from the first light interacting with the user; measuring emission counts of the emission signals over a period of time; causing to be determined at least one of (i) a level of the biomarker of the user and (ii) a change in the levels of the biomarker of the user over the period of time based on the measured emission counts; and generating a notification indicative of the at least one of (i) a level of the biomarker of the user and (ii) a change in the levels of the biomarker of the user.

Light can be emitted by, for example, an LED or LEDs. Emission counts can be measured by, for example, a photodetector or photodetectors. LEDs and photodetectors can be arranged such that a single given LED is paired with a single given photodetector. For example, a device according to the present disclosure can include, for example, a first LED-photodetector pair and a second LED-photodetector pair. This is not a requirement, however, as multiple LEDs can be paired with a single photodetector. An LED can be a LED that emits light of a single wavelength. An LED can also, however, be an LED that emits light of multiple wavelengths. A photodetector can be a single-channel photodetector. A photodetector can also be a multi-channel photodetector.

As discussed herein, the level of the biomarker and/or the change in the levels of the biomarker of the user can be determined, for example, based on a ratio of emission counts. For example, such a ratio can be the ratio of (i) emission counts collected by a first photodetector that collects emissions related to illumination provided by a first LED (e.g., an LED emitting at 475 nm) to (ii) emission counts collected by a second photodetector that collects emissions related to illumination provided by a second LED (e.g., an LED emitting at 515 nm). A change in emission counts related to LED emission at 475 nm can be compared against a change in emission counts related to LED emission at 515 nm, which can be indicative of a change in the user's glucose level.

In some cases, emission counts received at a first light detector (PD) can be compared to emission counts received at a second light detector at, or relatively at, the same time. In some cases, emission counts received at a first light detector can be compared to emission counts received at a second light detector at a different time. In some cases, ratios of counts can be compared against one another. For example, a ratio of counts received at a first light detector and counts received at a second light detector can be compared against a ratio of emission counts received by a third light detector and emission counts received at a fourth light detector at, or relatively at, the same time. In some examples, a ratio of counts received at a first light detector and counts received at a second light detector can be compared against a ratio of emission counts received by a third light detector and emission counts received at a fourth light detector at different times. In some examples, a ratio of emission counts received by a first light detector and emission counts received at a second light detector can be compared against the ratio of emission counts received by the first light detector and emission counts received at the second light detector collected at a different time.
Aspect 2. The method of Aspect 1, wherein the biomarker comprises any one or more of glucose, lactate, ketones, opioid levels, or blood alcohol.
Aspect 3. The method of any of Aspects 1 and 2, wherein the first light is generated by a first light source of the mobile device.
Aspect 4. The method of any of Aspects 1 through 3, wherein the emission signals are collected by a first detector of the mobile device.
Aspect 5. The method of any of Aspects 1 through 4, wherein the causing comprises sending data corresponding to the emission counts to a cloud network. It should also be understood that data can be stored and/or processed locally.
Aspect 6. The method of any of Aspects 1 through 5, wherein the first wavelength comprises 400 nm to 1200 nm. As an example, a first LED can emit illumination at a first wavelength of from 400 nm to 1200 nm (which wavelength can be safely applied to a user).
Aspect 7. The method of any of Aspects 1 through 6, wherein the emission signals have a wavelength of from 350 nm to 1200 nm. As an example, application to a user of illumination at a first wavelength can effect an emission signal having a wavelength of 700 nm. The wavelength of the emission signal need not be the same as the wavelength of the first wavelength. For example, application to a user of illumination at a first wavelength can effect an emission signal having a wavelength that differs from the first wavelength.
Aspect 8. The method of any of Aspects 1 through 7, wherein the measuring the emission counts indicates an increase or decrease in the emission counts over time, and wherein the change in the levels of the biomarker comprises an increase in the levels of the biomarker.
Aspect 9. The method of any of Aspects 1 through 8, wherein the measuring the emission counts indicates an increase or decrease in the emission counts over time, and wherein the change in the levels of the biomarker comprises a decrease in the levels of the biomarker.
Aspect 10. The method of any of Aspects 1 through 9, further comprising: generating a second light comprising a second wavelength; collecting second emission signals resulting from the second light interacting with the user; measuring second emission counts of the second emission signals over the period of time; and wherein the causing to be determined the change in the levels of the biomarker of the user over the period of time is further based on the measured second emission counts.
Aspect 11. The method of Aspect 10, wherein the causing to be determined the change in the levels of the biomarker further comprises comparing an emission spectra of the emission counts with a second emission spectra of the second emission counts.
Aspect 12. The method of Aspect 11, wherein comparing the emission spectra of the emission counts with the second emission spectra of the second emission counts further comprises determining a change in a ratio between the emission spectra of the emission counts with the second emission spectra of the second emission counts.
Aspect 13. The method of any of Aspects 1 through 12, wherein the mobile device comprises a mobile phone or a wearable device.
Aspect 14. The method of any of Aspects 1 through 13, wherein the first wavelength is selected on the basis of the biomarker.
Aspect 15. The method of any of Aspects 1 through 14, wherein the first wavelength is identified by a trained model.
Aspect 16. The method of Aspect 10, wherein the second wavelength is identified by a trained model.
Aspect 17. A mobile device for determining levels of a biomarker of a user, comprising: an illumination train configured to emit light comprising a first wavelength; a detector train configured to collect emission signals resulting from the light interacting with the user; and a controller configured to: measure emission counts of the emission signals over a period of time; cause to be determined at least one of (i) a level of the biomarker of the user and (ii) a change in the levels of the biomarker of the user over the period of time based on the measured emission counts; and generate a notification indicative of the at least one of (i) a level of the biomarker of the user and (ii) a change in the levels of the biomarker of the user over the period of time.
Aspect 18. The mobile device of Aspect 17, wherein the illumination train comprises a first light source configured to emit light comprising the first wavelength.
Aspect 19. The mobile device of any of Aspects 17 and 18, wherein the illumination train is configured to emit light comprising a second wavelength.
Aspect 20. The mobile device of any of Aspects 17 through 19, wherein the illumination train comprises a second light source configured to emit light comprising the second wavelength.
Aspect 21. The mobile device of Aspect 19, wherein the detector train is configured to collect second emission counts resulting from the light comprising the second wavelength interacting with the user.
Aspect 22. The mobile device of any of Aspects 17 through 21, wherein the first wavelength is selected on the basis of the biomarker.
Aspect 23. The mobile device of Aspect 22, wherein the first wavelength is identified by a trained model.
Aspect 24. The mobile device of any of Aspects 17 through 23, wherein the second wavelength is identified by a trained model.
Aspect 25. The mobile device of any of Aspects 17 through 24, wherein the first wavelength comprises 400 nm to 1200 nm.
Aspect 26. The mobile device of any of Aspects 17 through 25, wherein the measuring the emission counts indicates an increase in the emission counts over time, and wherein the change in the levels of the biomarker comprises an increase in the levels of the biomarker.
Aspect 27. The mobile device of any of Aspects 17 through 26, wherein the measuring the emission counts indicates an increase in the emission counts over time, and wherein the change in the levels of the biomarker comprises a decrease in the levels of the biomarker.
Aspect 28. The mobile device of any of Aspects 17 through 27, wherein the causing to be determined the change in the levels of the biomarker further comprises comparing an emission spectra of the emission counts with a second emission spectra of the second emission counts.
Aspect 29. The mobile device of Aspect 28, wherein comparing the emission spectra of the emission counts with the second emission spectra of the second emission counts further comprises determining a change in a ratio between the emission spectra of the emission counts with the second emission spectra of the second emission counts.
Aspect 30. The mobile device of any of Aspects 17 through 29, wherein the mobile device comprises a mobile phone or a wearable device.
Aspect 31. A mobile device for determining levels of a biomarker of a user, comprising: an illumination train configured to emit light comprising a plurality of wavelengths; a detector train configured to collect emission signals resulting from the plurality of wavelengths interacting with the user; and a controller configured to: measure emission counts of the emission signals over a period of time; with a trained model, cause to be determined at least one of (i) a level of the biomarker of the user and (ii) a change in the levels of the biomarker of the user over the period of time based on the measured emission counts; and generate a notification indicative of the at least one of (i) a level of the biomarker of the user and (ii) a change in the levels of the biomarker of the user over the period of time.
Aspect 32. The mobile device of Aspect 31, wherein the trained model comprises a relation between emission counts of emission signals interacting with the user and a ground truth level of the biomarker of the user.
Aspect 33. The mobile device of any of Aspects 31 and 32, wherein the illumination train comprises a first emitter configured to emit a light of a first wavelength and a second emitter configured to emit light a second wavelength, the first and second wavelengths differing from one another.
Aspect 34. The mobile device of any of Aspects 31 through 33, wherein the illumination train comprises a plurality of emitters, each emitter configured to emit a light of a wavelength unique to that emitter.
Aspect 35. The mobile device of Aspect 33, wherein the controller is configured to effect operation fewer than all of the plurality of emitters.
Aspect 36. A method of operating a mobile device, comprising: causing operation of an illumination train configured to emit light comprising a plurality of wavelengths; causing operation of a detector train configured to collect emission signals resulting from the plurality of wavelengths interacting with a user; and causing operation of a controller configured to: measure emission counts of the emission signals over a period of time; with a trained model, cause to be determined at least one of (i) a level of a biomarker of the user and (ii) a change in the levels of the biomarker of the user over the period of time based on the measured emission counts; and generate a notification indicative of the at least one of (i) a level of the biomarker of the user and (ii) a change in the levels of the biomarker of the user over the period of time.

It should be understood that the disclosed technology can implemented on a range of mobile devices. For example, the disclosed technology can be implemented on various devices having various hardware. For example, a device can include two light sources (LEDs), three light sources, four light sources, and the light, each configured to emit light of a respective wavelength. In some examples, the device can include two light detectors (PDs), three light detectors, fourth light detectors, and the like, where each is configured to collect emission signals from light interacting with a user (e.g., at respective wavelengths). Thus, the processes described herein can accommodate various hardware characteristics that the implementing device may have, and the technology described herein is not necessarily limited to any particular hardware configuration. As an example, the disclosed technology can be implemented on an existing mobile device. The disclosed technology can be embodied as software or other instructions that can be implemented on various devices having various hardware.
Aspect 37. The method of Aspect 36, wherein the biomarker comprises any one or more of glucose, lactate, ketones, opioid levels, or blood alcohol.

1. A method for determining levels of a biomarker of a user by a mobile device, comprising:
generating a first light comprising a first wavelength;
collecting emission signals resulting from the first light interacting with the user;
measuring emission counts of the emission signals over a period of time;
causing to be determined at least one of (i) a level of the biomarker of the user and (ii) a change in the levels of the biomarker of the user over the period of time based on the measured emission counts; and
generating a notification indicative of the at least one of (i) a level of the biomarker of the user and (ii) a change in the levels of the biomarker of the user.

2. The method of claim 1, wherein the biomarker comprises any one or more of glucose, lactate, ketones, opioid levels, or blood alcohol.

3. The method of claim 1, wherein the first light is generated by a first light source of the mobile device.

4. The method of claim 1, wherein the emission signals are collected by a first detector of the mobile device.

5. (canceled)

6. The method of claim 1, wherein the first wavelength comprises 400 nm to 1200 nm.

7. The method of claim 1, wherein the emission signals have a wavelength of from 350 nm to 1200 nm.

8. (canceled)

9. (canceled)

10. The method of claim 1, further comprising:
generating a second light comprising a second wavelength;
collecting second emission signals resulting from the second light interacting with the user;
measuring second emission counts of the second emission signals over the period of time; and
wherein the causing to be determined the change in the levels of the biomarker of the user over the period of time is further based on the measured second emission counts.

11. The method of claim 10, wherein the causing to be determined the change in the levels of the biomarker further comprises comparing an emission spectra of the emission counts with a second emission spectra of the second emission counts.

12. (canceled)

13. The method of claim 1, wherein the mobile device comprises a mobile phone or a wearable device.

14. The method of claim 1, wherein the first wavelength is selected on the basis of the biomarker.

15. (canceled)

16. (canceled)

17. A mobile device for determining levels of a biomarker of a user, comprising:
an illumination train configured to emit light comprising a first wavelength;
a detector train configured to collect emission signals resulting from the light interacting with the user; and
a controller configured to:
measure emission counts of the emission signals over a period of time;
cause to be determined at least one of (i) a level of the biomarker of the user and (ii) a change in the levels of the biomarker of the user over the period of time based on the measured emission counts; and
generate a notification indicative of the at least one of (i) a level of the biomarker of the user and (ii) a change in the levels of the biomarker of the user over the period of time.

18. The mobile device of claim 17, wherein the illumination train comprises a first light source configured to emit light comprising the first wavelength.

19. The mobile device of claim 17, wherein the illumination train is configured to emit light comprising a second wavelength.

20. (canceled)

21. The mobile device of claim 19, wherein the detector train is configured to collect second emission counts resulting from the light comprising the second wavelength interacting with the user.

22. (canceled)

23. (canceled)

24. (canceled)

25. The mobile device of claim 17, wherein the first wavelength comprises 400 nm to 1200 nm.

26. (canceled)

27. (canceled)

28. The mobile device of claim 21, wherein the causing to be determined the change in the levels of the biomarker further comprises comparing an emission spectra of the emission counts with a second emission spectra of the second emission counts.

29. The mobile device of claim 28, wherein comparing the emission spectra of the emission counts with the second emission spectra of the second emission counts further comprises determining a change in a ratio between the emission spectra of the emission counts with the second emission spectra of the second emission counts.

30. The mobile device of claim 17, wherein the mobile device comprises a mobile phone or a wearable device.

31. A mobile device for determining levels of a biomarker of a user, comprising:
an illumination train configured to emit light comprising a plurality of wavelengths;
a detector train configured to collect emission signals resulting from the plurality of wavelengths interacting with the user; and
a controller configured to:
measure emission counts of the emission signals over a period of time; with a trained model, cause to be determined at least one of (i) a level of the biomarker of the user and (ii) a change in the levels of the biomarker of the user over the period of time based on the measured emission counts; and
generate a notification indicative of the at least one of (i) a level of the biomarker of the user and (ii) a change in the levels of the biomarker of the user over the period of time.

32. (canceled)

33. The mobile device of claim 31, wherein the illumination train comprises a first emitter configured to emit a light of a first wavelength and a second emitter configured to emit light of a second wavelength, the first and second wavelengths differing from one another.

34-37. (canceled)
